(19) United States
(12) Patent Application Publication
(10) Pub. No.: US 2010/0034787 A1
(43) Pub. Date: Feb. 11, 2010

(54) COMPOSITION AND METHODS FOR PROMOTING HAIR GROWTH

(75) Inventor: Gail K. Naughton, San Diego, CA (US)

Correspondence Address: DLA PIPER LLP (US), 4365 EXECUTIVE DRIVE, SUITE 1100, SAN DIEGO, CA, 92121-2133, US

(73) Assignee: Histogen, Inc., San Diego, CA (US)

(21) Appl. No.: 12/580,122

(22) Filed: Oct. 15, 2009

Related U.S. Application Data
(63) Continuation of application No. 11/556,995, filed on Nov. 6, 2006, now abandoned.
Application No. 11/556,995 is a continuation of application No. 11/217,121, filed on Aug. 30, 2005, now abandoned.
(60) Provisional application No. 60/691,731, filed on Jun. 17, 2005.
(60) Provisional application No. 60/606,072, filed on Aug. 30, 2004.

(51) Int. Cl. A61K 35/12

(52) U.S. Cl. 424/93.7

(57) ABSTRACT

The present disclosure provides compositions and methods for promoting the growth of hair for cosmetic purposes as well as for treating disorders of hair growth. The compositions are conditioned media obtained from a three dimensional tissue that produces a combination of cellular factors effective to induce, promote, or enhance hair growth.

### 1. CROSS REFERENCES TO RELATED APPLICATIONS

This application is a continuation of U.S. application Ser. No. 11/556,995, filed Nov. 6, 2006, which is a continuation of U.S. application Ser. No. 11/217,121, filed Aug. 30, 2005, which claims the benefit of U.S. Provisional Application No. 60/691,731, filed Jun. 17, 2005, and U.S. Provisional Application No. 60/606,072, filed Aug. 30, 2004, the disclosures of which are incorporated herein by reference in their entireties.

### 2. BACKGROUND

The hair follicles of mammals develop from extensions of the embryonic epidermis that differentiate into three different layers of the mature hair. The central layer forms the hair shaft while the outer most layer forms the outer root sheath. The middle cylinder forms the inner root sheath that guides the hair shaft outward from the epidermis. At the base of the hair follicle is the dermal papilla, a pear shaped structure formed by a group of fibroblast cells derived from the mesoderm. The dermal papilla directs the embryonic generation of the hair follicle and also controls the regeneration of the hair follicle throughout its lifecycle. Thickness of the hair fiber correlates with the size of the dermal papilla. A basement membrane or basement lamina demarcates the dermal papilla cells from the hair fiber/sheath cells.

Normal mature hair follicles undergo a regenerative cycle defined by a growth stage (anagen), a degenerative stage (catagen), a resting stage (telogen), and a shedding stage (exogen). Anagen is the phase of hair follicle growth extending from the telogen stage to the beginning of the catagen stage and involves regrowth of the cycling part of the hair follicle. In anagen, dermal papilla fibroblasts secrete numerous growth factors that maintain active proliferation and differentiation of keratinocytes of the proximal hair bulb that forms the hair fiber. The length of the anagen phase, which may be further subdivided into six subphases (Anagen I-VI), is limited and is determinative of the hair shaft length. A longer anagen phase produces longer hair fibers. Anagen may be initiated in some instances by wounding of the hair follicle by plucking, vigorous shaving, or chemical insult (e.g., depilatory agents).

After the anagen stage, follicle growth stops and is followed by the catagen stage, at which time the fibroblasts retract from the basement membrane and the size of the papilla decreases. A decline in secretion of growth factors by the dermal papilla results in the reduction of proliferation and differentiation of hair matrix keratinocytes, resulting in cessation of hair shaft production. Epithelial cell death is prominent within the regressing follicle. At the end of catagen, follicular elements are lost around the papilla fibroblasts. As the hair follicle transitions to the telogen stage, the remaining hair takes on a club-shaped appearance with a small bud of the epithelial column being present at the follicle base. The telogen follicle rests in the dermis above the group of papilla fibroblasts. There are no further changes in the hair follicle until reinitiation of anagen.

A variety of conditions lead to hair loss and although the effect is primarily cosmetic, there is an adverse psychological impact on the affected patients. Because of this negative impact on body image, society expends substantial financial resources for various pharmacological agents, cosmetic treatments, surgical procedures, and prosthetic articles to counteract hair loss. Current pharmacological treatments for hair loss include Minoxidil and Finasteride. Minoxidil appears to lengthen the duration of the anagen stage by increasing the blood supply to the follicle, but appears to have to no direct effect in stimulating hair follicle development or growth. Topical treatment with the drug must be carried out continuously because cessation of treatment results in reversion to the pretreatment hair loss pattern. Finasteride, also known as Propecia®, is a 5 α-reductase Type II inhibitor targeting the intracellular enzyme responsible for converting the androgen testosterone into dihydrotestosterone. The drug is beneficial for patients with androgenetic alopecia because the condition is associated with elevated levels of dihydrotestosterone, which is believed to shorten the anagen stage of the hair follicle development. Finasteride can, however, cause ambiguous genitalia in developing male fetuses, thus limiting its use to men. Like Minoxidil, treatment with finasteride must be continuous because cessation of treatment leads to gradual progression of the disorder.

Because of the limited effect of hair loss treatments such as Minoxidil and finasteride, it is desirable to find alternative treatments.

### 3. SUMMARY

The present disclosure provides methods and compositions for treating hair loss and disorders characterized by hair loss. In some aspects, the compositions comprise conditioned media made from a three dimensional tissue in which the cultured tissue produces growth factors that promote hair follicle development and hair growth. In other aspects, the compositions comprise three dimensional tissues dimensioned for or so dimensioned for tissue penetration such that the three dimensional tissues can be administered by injection or a catheter. The methods comprise administering intradermally or subcutaneously to a subject an effective amount of the compositions. In some aspects, these treatments for promoting hair growth may be used for cosmetic purposes to produce a fuller appearance, change the hair line, or enhance hair growth.

In other aspects, the compositions may be used to treat a variety of disorders or conditions leading to loss of hair. Disorders include various forms of alopecia, such as androgenetic alopecia, alopecia areata, chemotherapy induced alopecia, and radiation induced alopecia. In various embodiments, the compositions may be used singly or in combination with other agents affecting hair growth, such as inducers of skin vascularization and inhibitors of dihydrotestosterone synthesis. Exemplary agents for inducing skin vascularization include Minoxidil and VEGF. Exemplary agents for inhibiting dihydrotestosterone synthesis are finasteride and dutasteride.

In further aspects, the compositions may be used to promote development and differentiation of hair follicles and associated cells in organ cultures or cell culture systems. The methods generally comprise contacting an epidermal stem cell or a cultured hair follicle with the compositions of the conditioned media or three dimensional tissues. These cultures may find uses in screening for compounds affecting hair growth or as tools for identifying biological factors involved in regulating hair follicle development and differentiation.

In some aspects, the compositions comprise isolated growth factors made by the three dimensional tissue. In some embodiments, the compositions comprise isolated Wnt proteins produced by the cultured cells. The suite of Wnt proteins elaborated by the cultures may be used, as well as one or more of Wnt5a, Wnt7a, and Wnt11 isolated from the conditioned media. The Wnt proteins may be used independently or in combination with other agents affecting hair growth (e.g., VEGF, Minoxidil, finasteride, etc.)

In other aspects, provided herein are kits comprising the compositions in various pharmaceutical formulations for cosmetic applications and for treatment of hair loss. The compositions may be provided in various dosage forms, such as injectable suspensions or lyophilizates for reconstitution with a suitable diluent for injection, and topical formulations for adjunctive administration. In other embodiments, the kits may further comprise other agents affecting hair growth, such as Minoxidil and/or finesteride.

### 4. BRIEF DESCRIPTION OF THE FIGURES

FIG. 1 shows the effect of three dimensional tissue conditioned medium on cell proliferation in four different epithelial cell types (Caco-2, NCI-H292, primary keratinocytes, and HT29). The conditioned medium enhanced cell growth in primary keratinocytes and NCI-H292 cells but not for the most part in Caco-2 or HT29 cells. Not all cell lines responded, and not all conditioned medium were effective.

FIG. 2 shows phase contrast microscopy of cells treated with conditioned media. Caco-2, NCI-H292 and epidermal keratinocyte (not shown) morphology is altered by the conditioned medium whereas HT-29 cells (not shown) were not affected. The apparent formation of dome like structure in the Caco-2 and NCI-H292 cell lines may indicate an enhancement or induction of differentiation, as mucin-production (a marker of both intestinal and respiratory epithelium) has been reported to lead to similar morphological changes in these cell lines as well as primary human intestinal and respiratory cells in culture.

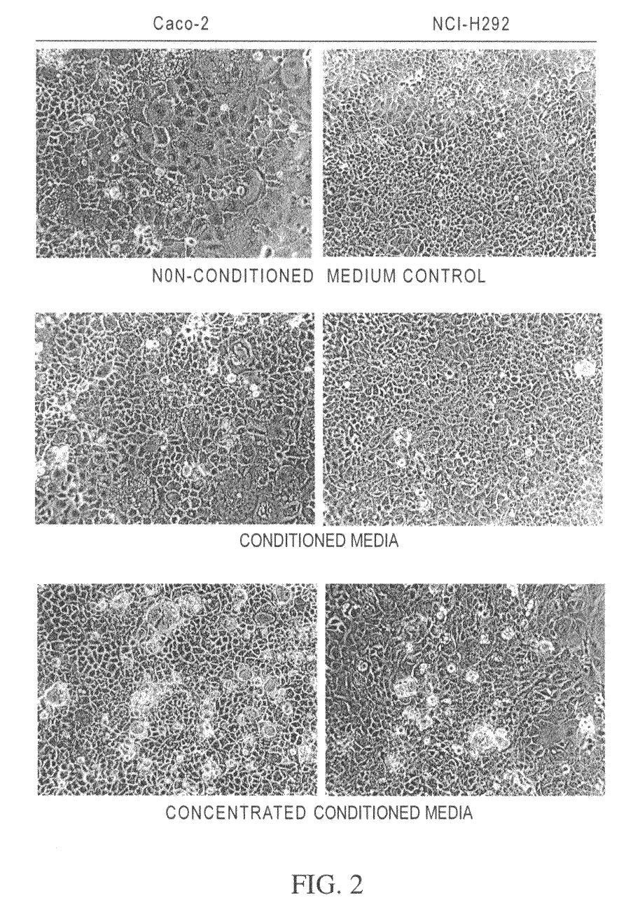

FIG. 3 shows analysis by immunofluorescence of adherens (ZO-1) and tight junction marker (claudin-1) in Caco-2 cells placed on collagen coated glass slides and treated with conditioned media from three dimensional stromal cell tissues. There is discontinuous staining for ZO-1 in the control medium panel (white arrow), and the junctional localization of claudin-1 in all the three dimensional tissue conditioned medium treated panels (dashed white arrow).

FIG. 4 shows the morphological changes in organotypic, high density microporous membrane cultures treated with conditioned medium as examined by transmission electron microscopy. A tissue section is shown for control and three-dimensional stromal tissue conditioned medium treated Caco-2 cells. There is increased overall thickness, more columnar shape, and an increase in intracellular spaces in the cells treated with the three dimensional tissue conditioned medium. These are all characteristics of normal differentiation of these cell types.

FIG. 5 shows a higher magnification transmission electron microscope analysis of effects of three dimensional stromal tissue conditioned medium <10 kD permeate on Caco-2 cells in high density organotypic cultures. There is an increase in cellular processes, microvilli, and mitochondrial location (apical in the three dimensional stromal tissue conditioned medium sample). Tight junctions were less frequent in the three dimensional tissue conditioned medium sample than the control.

FIG. 6 is a TEM analysis of three dimensional stromal tissue conditioned medium <10 kD permeate on Caco-2 cells in high density organotypic cultures showing effects on cellular processes, apical microvilli, and dense glycogen deposits.

FIG. 7 shows the effect of injecting conditioned medium on stimulation of hair growth in adult C57B1/6 mice. Hair growth was examined 30 days after injection.

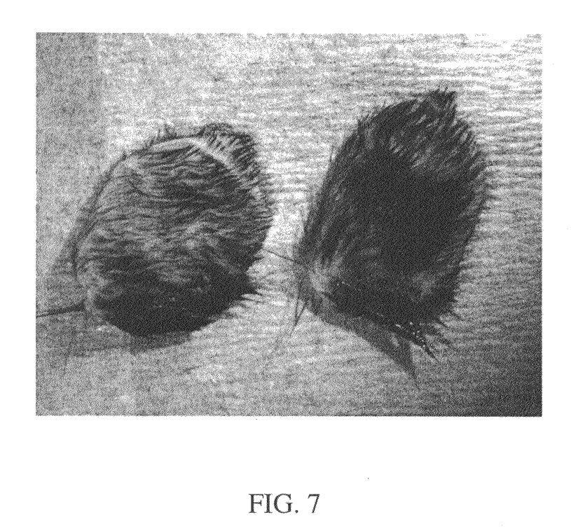

FIG. 8 shows C57B1/6 mice treated with conditioned medium.

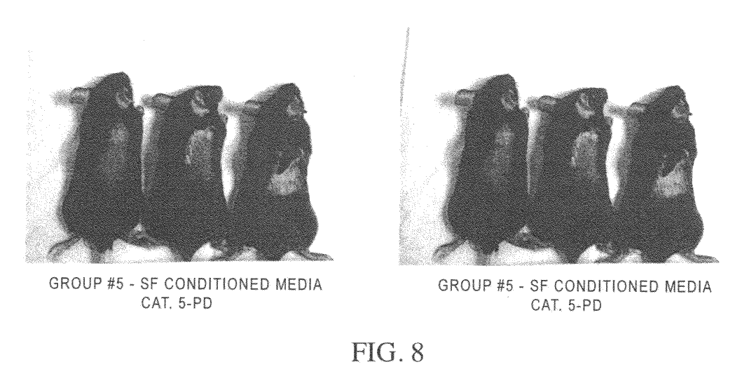

FIG. 9 shows skin pigmentation changes after 30 days from injected conditioned medium.

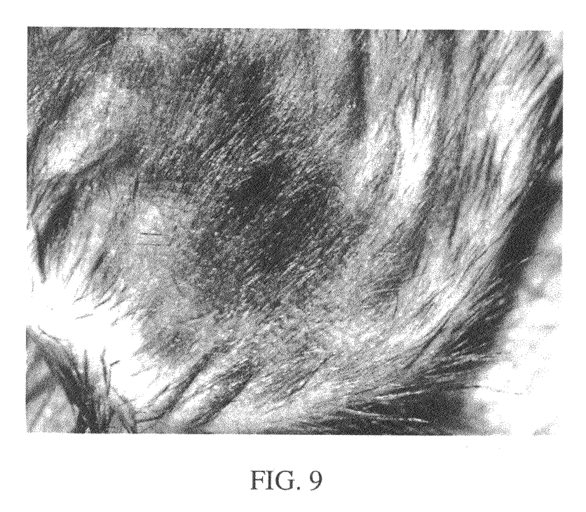

FIG. 10 shows histological examination of the skin in animals treated with conditioned medium.

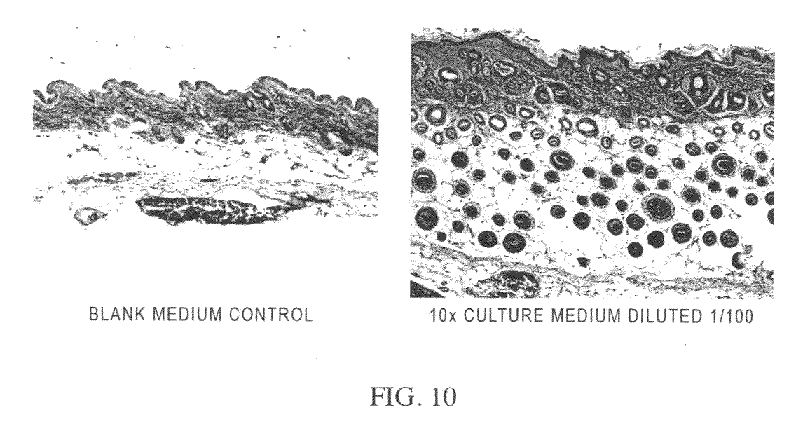

FIG. 11 is a histological comparison (using trichome stain) after 14 days showing that injection of conditioned medium induced hair follicles after 14 days in mice receiving 10× conditioned medium diluted 1/100 as compared to blank medium controls.

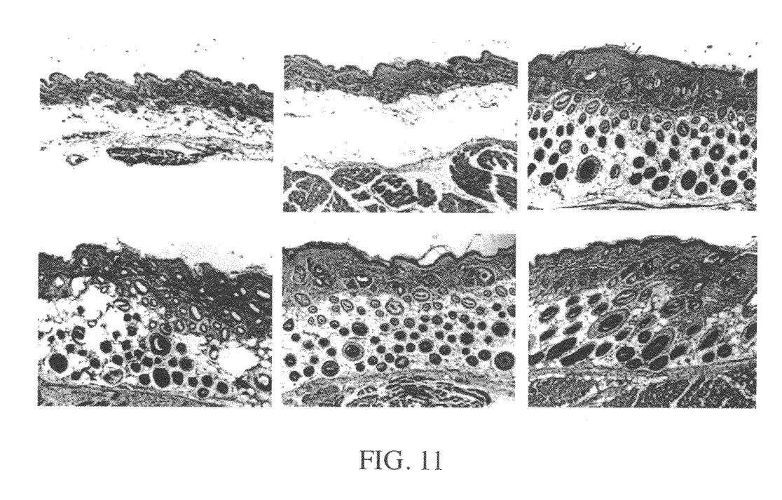

FIG. 12 shows that hair follicle anagen induction by the conditioned medium is confined to the site of injection.

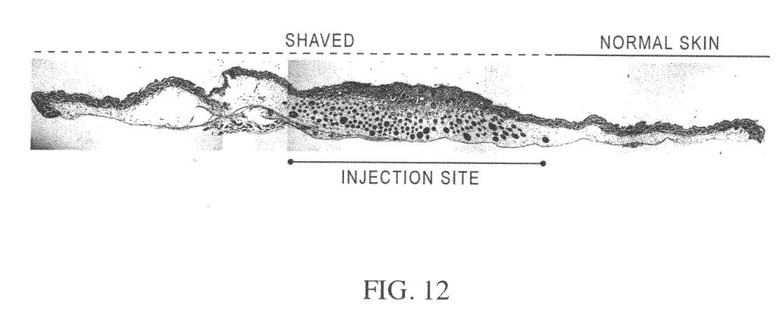

FIG. 13 shows cross sections of follicles induced by injection of conditioned medium. The follicles exhibit normal anagen morphology, including connective tissue sheath, outer root sheath, inner root sheath, cortex, matrix, and dermal papilla.

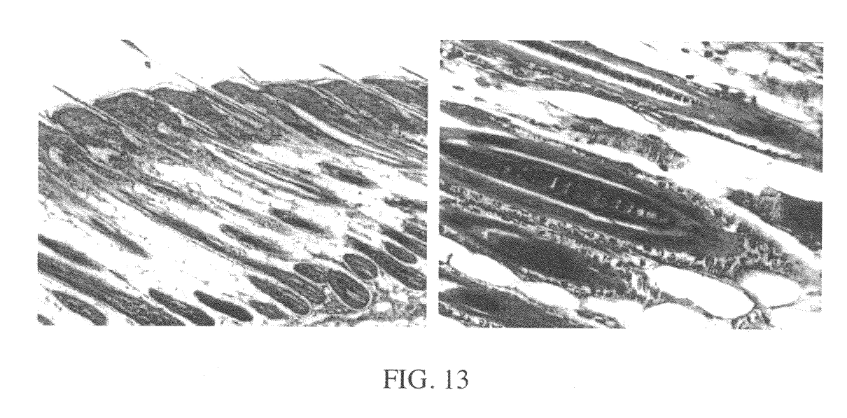

FIG. 14 shows Keratin 15 and Keratin 10 expression in conditioned medium-induced hair follicles, evidencing of normal, mature hair follicles.

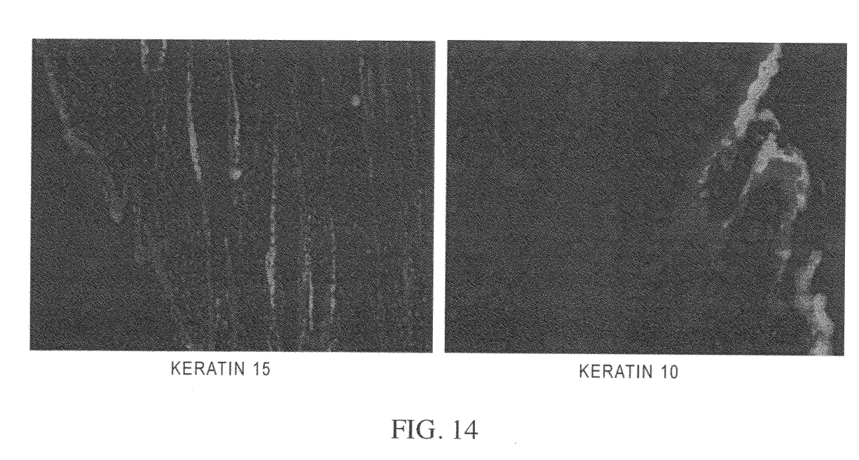

FIG. 15 shows Wnt signaling in epidermal keratinocytes in vitro. Nuclear translocation of β-catenin is induced by conditioned medium, providing evidence that the conditioned medium contains Wnt proteins.

FIG. 16 shows a histological evaluation of hair follicle formation in adult SCID mice. Left panel is a tissue section from an animal injected with blank medium control and the right panel shows a tissue section from an animal injected with three dimensional tissue conditioned medium. Adult SCID mouse were injected once subcutaneously (SQ) and the animals examined after 10 days.

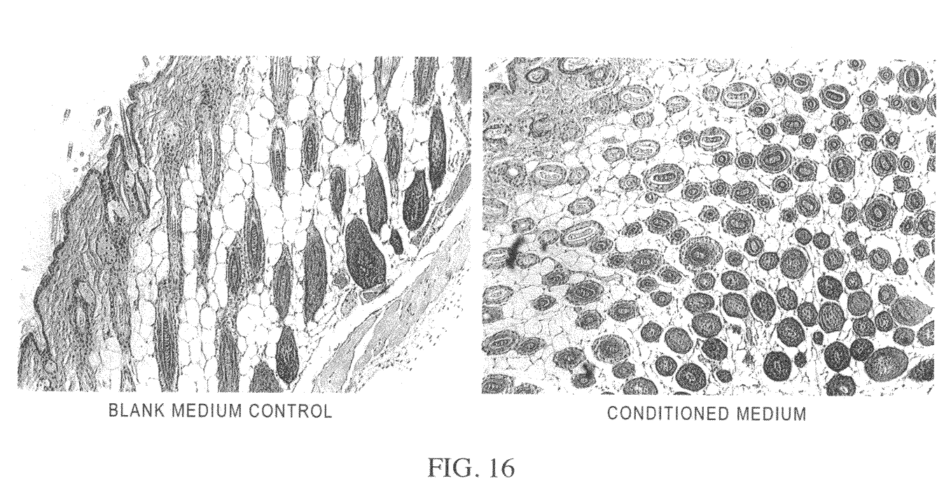

FIG. 17 shows quantification of follicular structures induced by three dimensional stromal tissue conditioned medium after 14 days. The y-axis is number of follicle-like structures in the histology field.

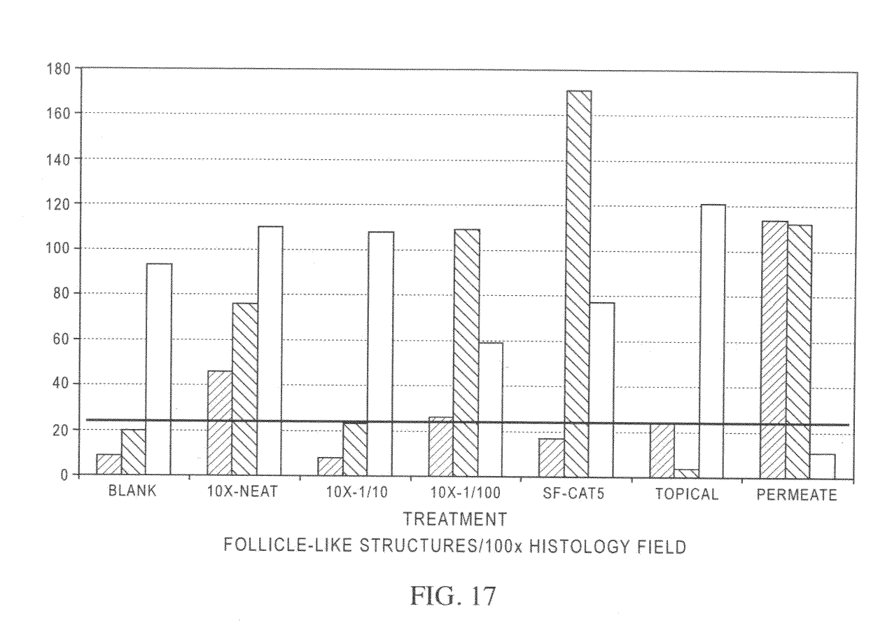

FIG. 18 shows combined data quantifying the follicular structures (N=6).

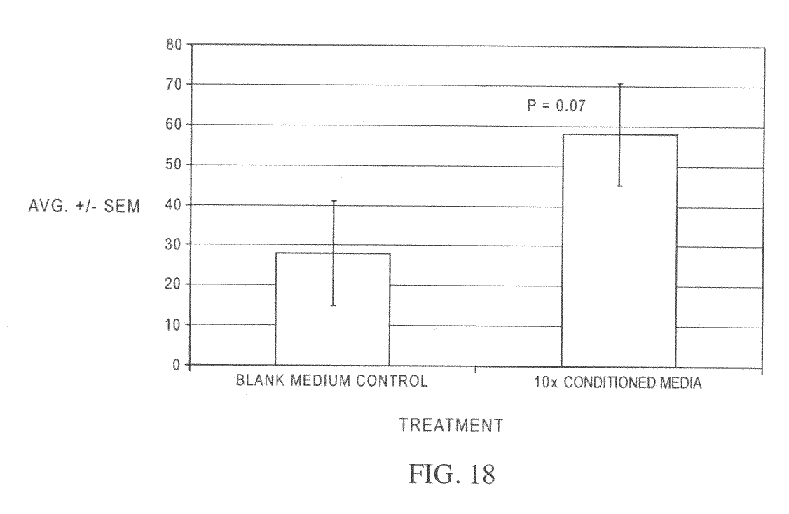

### 5. DETAILED DESCRIPTION

The present disclosure provides compositions and methods for promoting hair growth. It is shown herein that administering a composition of conditioned media made from a three dimensional tissue cells promotes hair growth. Unlike topical treatments with conditioned medium, which result in some changes to skin morphology, subcutaneous or intradermal administration of such conditioned medium appears to induce development of hair follicles and promote hair growth. Without being limited by theory, the three dimensional tissue appears to secrete a combination of growth factors, including a characteristic group of Wnt proteins, that may recruit and stimulate differentiation of epidermal stem cells into hair follicles. The factors may mimic inductive signals from dermal papillar cells present during fetal development such that competent adult epidermal stem cells may respond to the factors by forming new follicles rather than simply promoting hair follicle cycling in existing hair follicles.

Generally, the methods herein comprise administering intradermally or subcutaneously to a subject an effective amount of a composition comprising a conditioned media made from a three dimensional tissue or administering the three dimensional tissue itself. In other embodiments, the administered compositions comprise Wnt proteins isolated from the conditioned media of the three dimensional tissues.

### 5.1 Three Dimensional Scaffolds

In various embodiments, the conditioned medium capable of promoting the growth of hair follicles is obtained from a three dimensional tissue. As used herein, “conditioned media” refers to culture media in which cells have been cultured and into which the cells have secreted active agent(s) to sufficient levels to display a desired biological activity or activities. In some embodiments, the “conditioned media” is characterized by a fingerprint or repertoire of cell-produced factors present in the media.

In various embodiments, the cultured cells are supported by a framework (synonymously “scaffold”) comprised of a biocompatible, non-living material. The scaffold or framework may be of any material and/or shape that: (a) allows cells to attach to it (or can be modified to allow cells to attach to it); and (b) allows cells to grow in more than one layer (i.e. form a three dimensional tissue). In other embodiments, a substantially two-dimensional sheet or membrane may be used to culture cells that are sufficiently three dimensional in form such that the conditioned media displays the desired hair promoting activity. Descriptions for cell cultures using a three dimensional framework are described in U.S. Pat. Nos. 6,372,494; 6,291,240; 6121,042; 6,022,743; 5,962,325; 5,858,721; 5,830,708; 5,785,964; 5,624,840; 5,512,475; 5,510,254; 5,478,739; 5,443,950; and 5,266,480; all publications incorporated herein by reference in their entirety. Commercial embodiments are available under the tradename Dermagraft® (Smith & Nephew, Indianapolis, Ind., USA).

In some embodiments, the biocompatible material is formed into a three-dimensional structure or scaffold, where the structure has interstitial spaces for attachment and growth of cells into a three dimensional tissue. The openings and/or interstitial spaces of the framework in some embodiments are of an appropriate size to allow the cells to stretch across the openings or spaces. Maintaining actively growing cells stretched across the framework appears to enhance production of the repertoire of growth factors responsible for the activities described herein. If the openings are too small, the cells may rapidly achieve confluence but be unable to easily exit from the mesh. These trapped cells may exhibit contact inhibition and cease production of the appropriate factors necessary to support proliferation and maintain long term cultures. If the openings are too large, the cells may be unable to stretch across the opening, which may lead to a decrease in stromal cell production of the appropriate factors necessary to support proliferation and maintain long term cultures. Typically, the interstitial spaces are at least about 140 um, at least about 150 um, at least about 180 um, at least about 200 um, or at least about 220 um. However, depending upon the three-dimensional structure and intricacy of the framework, other sizes are permissible. Any shape or structure that allows the cells to stretch and continue to replicate and grow for lengthy time periods may function to elaborate the cellular factors in accordance with the methods herein.

In some embodiments, the three dimensional framework is formed from polymers or threads that are braided, woven, knitted or otherwise arranged to form a framework, such as a mesh or fabric. The materials may also be formed by casting of the material or fabrication into a foam, matrix, or sponge-like scaffold. In other embodiments, the three dimensional framework is in the form of matted fibers made by pressing polymers or other fibers together to generate a material with interstitial spaces. The three dimensional framework may take any form or geometry for the growth of cells in culture as long as the conditioned media produced therefrom displays hair growth promoting activities described herein. Thus, other forms of the framework, as further described below, may suffice for generating the appropriate conditioned medium.

A number of different materials may be used to form the scaffold or framework. These materials include non-polymeric and polymeric materials. Polymers, when used, may be any type of polymer, such as homopolymers, random polymers, copolymers, block polymers, coblock polymers (e.g., di, tri, etc.), linear or branched polymers, and crosslinked or non-crosslinked polymers. Non-limiting examples of materials for use as scaffolds or frameworks include, among others, glass fibers, polyethylenes, polypropylenes, polyamides (e.g., nylon), polyesters (e.g. dacron), polystyrenes, polyacrylates, polyvinyl compounds (e.g., polyvinylchloricie; PVC), polycarbonates, polytetrafluorethylenes (PTFE; TEFLON), thermanox (TPX), nitrocellulose, polysaacharides (e.g., celluloses, chitosan, agarose), polypeptides (e.g., silk, gelatin, collagen), polyglycolic acid (PGA), and dextran.

In some embodiments, the framework may be made of materials that degrade over time under the conditions of use. Biodegradable also refers to absorbability or degradation of a compound or composition when administered in vivo or under in vitro conditions. Biodegradation may occur through the action of biological agents, either directly or indirectly. Non-limiting examples of biodegradable materials include, among others, polylactide, polyglycolide, poly(trimethylene carbonate), poly(lactide-co-glycolide) (i.e., PLGA), polyethylene terephtalate (PET), polycaprolactone, catgut suture material, collagen (e.g., equine collagen foam), polylactic acid, or hyaluronic acid. For example, these materials may be woven into a three-dimensional framework such as a collagen sponge or collagen gel.

In other embodiments, where the cultures are to be maintained for long periods of time, cryopreserved, and/or where additional structural integrity is desired, the three dimensional framework may be comprised of a nonbiodegradable material. As used herein, a nonbiodegradable material refers to a material that does not degrade or decompose significantly under the conditions in the culture medium. Exemplary nondegradable materials include, as non-limiting examples, nylon, dacron, polystyrene, polyacrylates, polyvinyls, polytetrafluoroethylenes (PTFE), expanded PTFE (ePTFE), and cellulose. An exemplary nondegrading three dimensional framework comprises a nylon mesh, available under the tradename Nitex®, a nylon filtration mesh having an average pore size of 140 μm and an average nylon fiber diameter of 90 μm (#3-210/36, Tetko, Inc., N.Y.).

In other embodiments, the three dimensional scaffold or framework is a combination of biodegradeable and non-biodegradeable materials. The non-biodegradable material provides stability to the three dimensional scaffold during culturing while the biodegradeable material allows formation of interstitial spaces sufficient for generating cell networks that produce the cellular factors sufficient for promoting hair growth. The biodegradable material may be coated onto the non-biodegradable material or woven, braided or formed into a mesh. Various combinations of biodegradable and non-biodegradable materials may be used. An exemplary combination is poly(ethylene therephtalate) (PET) fabrics coated with a thin biodegradable polymer film, poly[D-L-lactic-co-glycolic acid), in order to obtain a polar structure.

In various embodiments, the scaffold or framework material may be pre-treated prior to inoculation with cells to enhance cell attachment. For example, prior to inoculation with cells, nylon screens in some embodiments are treated with 0.1 M acetic acid, and incubated in polylysine, fetal bovine serum, and/or collagen to coat the nylon. Polystyrene could be similarly treated using sulfuric acid. In other embodiments, the growth of cells in the presence of the three-dimensional support framework may be further enhanced by adding to the framework or coating it with proteins (e.g., collagens, elastin fibers, reticular fibers), glycoproteins, glycosaminoglycans (e.g., heparan sulfate, chondroitin-4-sulfate, chondroitin-6-sulfate, dermatan sulfate, keratan sulfate, etc.), fibronectins, and/or glycopolymer (poly[N-p-vinylbenzyl-D-lactoamide], PVLA) in order to improve cell attachment. Treatment of the scaffold or framework is useful where the material is poor substrate for the attachment of cells.

In other embodiments, the scaffold or framework for generating the cultured three dimensional tissues are dimensioned for or so dimensioned as to permit penetration into tissues. These compositions elaborate the suite or repertoire growth factors that promote hair growth while being administrable by minimally invasive methods, such as by injection or a catheter. For these embodiments, the conditioned medium made from these three dimensional tissues or the three dimensional tissue itself may be administered to promote hair growth. Various embodiments of these three dimensional tissues are described in U.S. patent application Ser. No. 11/216,580, entitled “Cultured Three Dimensional Tissues and Uses Thereof,” filed concurrently herewith, the disclosure of which is incorporated herein by reference in its entirety.

In some tissue penetrating embodiments, the framework for the cell cultures comprises particles that, in combination with the cells, form a three dimensional tissue. The cells attach to the particles and to each other to form a three dimensional tissue. The complex of the particles and cells is of sufficient size to be administered into tissues or organs, such as by injection or catheter. As used herein, a “micropartjcle” refers to a particle having size of nanometers to micrometers, where the particles may be any shape or geometry, being irregular, non-spherical, spherical, or ellipsoid. Microparticles encompass microcapsules, which are microparticles with one or more coating layers. In some embodiments, the microparticles comprise microspheres. As used herein “microspheres” refer to microparticles with a spherical geometry. A microsphere, however, need not be absolutely spherical, as deviations are permissible for generating the three dimensional tissues.

The size of the microparticles suitable for the purposes herein can be determined by the person skilled in the art. In some embodiments, the size of microparticles suitable for the three dimensional tissues may be those administrate by injection. In some embodiments, the microparticles have a particle size range of at least about 1 μm, at least about 10 μm, at least about 25 μm, at least about 50 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1000 μm. The characteristics and size of the microparticles can be readily determined using a variety of techniques, such as scanning electron microscopy, light scattering, or differential scanning calorimetry.

In some embodiments in which the microparticles are made of biodegradable materials, the particles are made to have a defined half-life under a defined biological condition. “Mean half life” as used in the context of microparticles refers to the mean time required for the particles to degrade to half the initial mass of a microparticle. The half-life of the microparticles may vary depending on various parameters, including, among others, type of biodegradable polymers, the polymer porosity (e.g., porous or nonporous), molecular weight of the polymers, microparticle geometry, and level of polymer crosslinking. Choosing microparticles with a short or long half life may be varied by the practitioner depending on the frequency of administration, the longevity of the cells following administration, and the time that the three dimensional tissue is effective in producing the desired effect, such as elaboration of a suite of growth factors. Thus in some embodiments, the microparticles in the three dimensional tissues have a mean half-life of about 14 days, a mean half-life of about 28 days, a mean half-life of about 90 days, or a mean half-life of about 180 days. As will be apparent to the skilled artisan, the half-life may be made shorter or longer to achieve the desired therapeutic properties of the compositions.

In some embodiments, to vary its half life, microparticles comprising two or more layers of different biodegradable polymers may be used. In some embodiments, at least an outer first layer has biodegradable properties for forming the three dimensional tissues in culture, while at least a biodegradable inner second layer, with properties different from the first layer, is made to erode when administered into a tissue or organ.

In some embodiments, the microparticles are porous microparticles. Porous microparticles refer to microparticles having interstices through which molecules may diffuse in or out from the microparticle. In other embodiments, the microparticles are non-porous microparticles. A nonporous microparticle refers to a microparticle in which molecules of a select size do not diffuse in or out of the microparticle.

Microparticles for use in the compositions are biocompatible and have low or no toxicity to cells. Suitable microparticles may be chosen depending on the tissue to be treated, type of damage to be treated, the length of treatment desired, longevity of the cell culture in vivo, and time required to form the three dimensional tissues. The microparticles may comprise various polymers, natural or synthetic, charged (i.e., anionic or cationic) or uncharged, biodegradable, or nonbiodegradable. The polymers may be homopolymers, random copolymers, block copolymers, graft copolymers, and branched polymers.

In some embodiments, the microparticles comprise non-biodegradable microparticles. Non-biodegradable microcapsules and microparticles include, but not limited to, those made of polysulfones, poly(acrylonitrile-co-vinyl chloride), ethylene-vinyl acetate, hydroxyethylmethacrylate-methyl-methacrylate copolymers. These are useful to provide tissue bulking properties or in embodiments where the microparticles are eliminated by the body.

In some embodiments, the microparticles comprise degradable scaffolds. These include microparticles made from naturally occurring polymers, non-limiting example of which include, among others, fibrin, casein, serum albumin, collagen, gelatin, lecithin, chitosan, alginate or poly-amino acids such as poly-lysine. In other embodiments, the degradable microparticles are made of synthetic polymers, non-limiting examples of which include, among others, polylactide (PLA), polyglycolide (PGA), poly(lactide-co-glycolide) (PLGA), poly(caprolactone), polydioxanone trimethylene carbonate, polyhybroxyalkonates (e.g., poly(hydroxybutyrate), poly(ethyl glutamate), poly(DTH iminocarbony(bisphenol A iminocarbonate), poly(ortho ester), and polycyanoacrylates.

In some embodiments, the microparticles comprise hydrogels, which are typically hydrophilic polymer networks filled with water. Hydrogels have the advantage of selective trigger of polymer swelling. Depending on the composition of the polymer network, swelling of the microparticle may be triggered by a variety of stimuli, including pH, ionic strength, thermal, electrical, ultrasound, and enzyme activities. Non-limiting examples of polymers useful in hydrogel compositions include, among others, those formed from polymers of poly (lactide-co-glycolide); poly(N-isopropylacrylamide); poly(methacrylic acid-g-polyethylene glycol); polyacrylic acid and poly(oxypropylene-co-oxyethylene)glycol; and natural compounds such as chrondroitan sulfate, chitosan, gelatin, fibrinogen, or mixtures of synthetic and natural polymers, for example chitosan-poly(ethylene oxide). The polymers may be crosslinked reversibly or irreversibly to form gels adaptable for forming three dimensional tissues (see, e.g., U.S. Pat. Nos. 6,451,346; 6,410,645; 6,432,440; 6,395,299; 6,361,797; 6,333,194; 6,297,337; Johnson et al., 1996, Nature Med. 2:795; incorporated by reference in their entireties).

In some embodiments, another type of particles useful in the compositions and methods of this disclosure comprise nanoparticles, which are generally microparticles of about 1 um or less in diameter or size. In some embodiments, the nanoparticles have a particle size range of at least about 10 nm, at least about 25 nm, at least about 50 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1000 nm. Nanoparticles are generally made from amphiphilic diblock, triblock, or multiblock copolymers as is known in the art. Polymers useful in forming nanoparticles include, but are limited to, polylactide (PLA; see Zambaux et al., 1999, J. Control Release 60:179-188), polyglycolide, poly(lactide-co-glycolide), blends of poly(lactide-co-glycolide) and polycarprolactone, diblock polymer poly(1-leucine-block-1-glutamate), diblock and triblock poly(lactic acid) (PLA) and poly(ethylene oxide) (PEO) (De Jaeghere et al., 2000, Pharm. Dev. Technol. 5:473-83), acrylates, arylamides, polystyrene. As described for microparticles, nanoparticles may be non-biodegradable or biodegradable. Nanoparticles may be also be made from poly(alkylcyanoacrylale), for example poly (butylcyanoacrylate), in which proteins are absorbed onto the nanoparticles and coated with surfactants (e.g., polysorbate 80).

Various methods for making microparticles are well known in the art, including, among others, solvent removal process (see, e.g., U.S. Pat. No. 4,389,330), emulsification and evaporation (Maysinger et al., 1996, Exp. Neuro. 141:47-56; Jeffrey et al., 1993, Pharm. Res. 10:362-68), spray drying, and extrusion methods. Methods for making nanoparticles are similar to those for making microparticles and include, among others, emulsion polymerization in continuous aqueous phase, emulsification-evaporation, solvent displacement, and emulsification-diffusion techniques (see Kreuter, 1991. J., “Nano-particle Preparation and Applications.” in Microcapsules and nanoparticles in medicine and pharmacy, pg. 125-148, (M. Donbrow, ed.) CRC Press, Boca Raton, Fla., incorporated by reference).

In other embodiments of tissue penetrating compositions, the scaffold or framework of the three dimensional tissue is made from a nonwoven network of biodegradable, biocompatible filaments that form particulate structures when incubated with cells in a culture medium. Generally, the nonwoven filaments comprise matted natural or synthetic polymeric or fibrous material formed into a three dimensional scaffold, such as in the form of a web, fell, or pulp. The nonwoven framework provides a three dimensional structure that allows cells to proliferate and form cell-cell contacts to generate a tissue-like structure and elaborate the suite of growth factors having the desired biological properties. The fibers act as struts, defining the boundaries of the interstitial spaces; cells attach to the fibers and proliferate to fill the void spaces in the nonwoven network. While not being bound by any theory of action, the particulate composition of the matted fibers and cells appears to form as the fibers or polymers degrade under culture conditions and pockets or isolated masses of nonwoven filaments and cells detach from the original network of fibers or polymers.

The nonwoven network may be formed in some embodiments by compressing intertwined or entangled fibers or polymers. In other embodiments, the filament junctions or crosspoint may be bonded to provide mechanical strength and/or a three dimensional lattice. Although the scaffold or framework is nonwoven, it is to be understood that two or more plies of nonwoven fabric may be attached together by stitching, or use of a binder, such as an adhesive. The layers or plies are typically positioned in a juxtaposed or a surface-to-surface relationship. Different density of matted fibers may be used to alter the properties of the three dimensional framework, for instance, to add mechanical strength or increase the time required for degradation of the scaffold (see, e.g., U.S. Pat. No. 6,077,526).

The fibers may be of uniform length or random length and may be made from natural or synthetic fibers, or combinations thereof. The filaments may also comprise a uniform diameter or may be comprised of filaments of differing diameters. In embodiments in which the nonwoven filaments comprise blends of compatible fibers, the mixtures may be fibers of differing mechanical strength, degradation rate, and/or adhesiveness. Filaments of shorter length may produce the particulate compositions with shorter culturing times but which dissipates (asters when administered in vivo while filaments of longer length may produce particulate compositions with longer culturing times but which dissipates more slowly upon administration (see, e.g., Wang et al., 1997, J. Biomater. Sci. Polymer Edn. 9(1):75-87. The choice of filaments to form the non-woven framework is readily determined by the person skilled in the art.

In some embodiments, the nonwoven three dimensional framework may further comprise non-biodegradable polymers, as further described below. Non-biodegradable polymers may be used to provide mechanical strength to and durability to the nonwoven network of biodegradable polymers. In some embodiments, the non-degradable polymers have lengths suitable for passage through an injection needle and/or allow formation of particulates of three dimensional tissues.

The nonwoven network of filaments may be made of various fibers or polymers, natural or synthetic. Biodegradable filaments for making the nonwoven three dimensional framework may employ fibers and polymers used to make other types of scaffold structures described herein. The polymers may be homopolymers, random copolymers, block copolymers, graft copolymers, and branched polymers. Non-limiting examples of biodegradable natural polymers include among others, catgut, elastin, fibrin, hyaluronic acid, cellulose derivatives, and collagen. Non-limiting examples of biodegradable synthetic polymers include, among others, polylactide, polyglycolide, poly(e-caprolactone), poly(trimethylene carbonate) and poly(p-dioxanone), and copolymers, such as poly(lactide-co-glycolide), poly(e-caprolactone-co-glycolide), poly(glycolide-co-trimethylene carbonate), poly(alkylene diglycolate), and polyoxaesters. Descriptions for the preparation of such polymers and fibers are provided in various reference works and publications, such as Sorensen et al., 1968, Preparative Methods of Polymer Chemistry, Wiley, NY; Biodegradable Polymers As Active Agent Delivery Systems, (Chasin et al., eds.) Marcel Dekker Inc., NY, 1997; and U.S. Pat. Nos. 6,866,860; 6,703,477; 5,348,700; 5,066,772; 4,481,353; 4,243,775; 4,429,080; and 4,157,357).

In some embodiments, the nonwoven three dimensional framework may comprise a combination of polymers (i.e., polymer blends) so long as they do not interfere with formation of the three dimensional tissues or the biodegradable characteristics of the compositions. Blends of the polymers may provide flexibility in providing the desired characteristics of particulate formation in culture, mechanical strength, durability when administered in vivo, and tissue bulking properties.

The nonwoven scaffold may be made by conventional techniques known in the art. Filaments, such a fibers or polymers of various lengths are made and then formed into a web or entangled matt, and the filaments optionally bonded within the web or matt by an adhesive or by mechanical frictional forces. For forming the particulate compositions, the nonwoven filaments are inoculated with the cells, as described below, and cultured in presence of the cells until portions of the filaments detach and form isolated or detached particles of scaffold and cells. In some embodiments, formation of injectable particulates may be accelerated by mechanical action. This may be carried out in various ways, such as by passing the compositions through an orifice (i.e., needle) or gentle mechanical shearing. Preparation of the compositions will be well within the capabilities of the skilled artisan.

In some embodiments, the three dimensional scaffold is formed from multiple filaments, polymers or fibers that are braided, twisted, or woven, or otherwise arranged into a cord or a thread like structure that can be administered or inserted into tissues or organs. The scaffold comprises interstitial spaces that allow cells to attach and proliferate to form a three dimensional culture of living cells. In some embodiments, the braided or woven thread is suitable for use as a surgical suture material.

The cord or suture may be made in a range of conventional forms or constructions to have the interstitial spaces for invasion and attachment of cells and their proliferation. As noted above, the openings and/or interstitial spaces of the cord scaffold should be of an appropriate size to allow the cells to stretch across the openings or spaces. Thus, the interstitial spaces in the braided framework are at least about 140 μm, at least about 150 μm, at least about 180 μm, at least about 200 μm, or at least about 220 μm.

In some embodiments, the filaments are woven to form a luminal space for the proliferation of cells. The internal luminal space, which is a void space prior its occupation by cells, may or may not be occupied by a core filament. Although the luminal space may comprise varying geometric structures, luminal spaces in the braided structures may be in the form of a tube that runs lengthwise along the cord or sheath. Where a core is present, the sheath forms a jacket around the core. Different types of braids are known in the art. A spiral braid having different braiding angles may be made into cords with different tensile strengths. The core, when present, can be of various constructions, including, among others, a single filament or multiple filaments (see, e.g., U.S. Pat. No. 6,045,571), twisted or plied, and comprise a material that is the same or different from the sheath.

The cord or suture may be made from various materials described above for preparing other three dimensional scaffolds and frameworks. Homopolymers, random copolymers, block copolymers, and branched polymers may be used to form the cords or sutures. Non-limiting examples of biodegradable materials include, among others, polylactide (PLA), polyglycolide (PGA), poly(lactide-co-glycolide) (PLGA), polyethylene terephtalate (PET), polycaprolactone, dioxanone, trimethylene carbonate (TMC), poly(alkylene oxalate), polyoxaesters, copolymers made of PGA/PLA/TMC or any combination thereof in any percent combination, catgut suture material, collagen (e.g., equine collagen foam), hyaluronic acid, and compatible mixtures or blends thereof (see, e.g., U.S. Pat. No. 6,632,802; Biomedical Polymers, (Shalaby et al., eds.) Verlag, 1994; U.S. Pat. No. 6,177,094; U.S. Pat. No. 5,951,997).

In other embodiments in which additional structural integrity, durability, and/or tensile strength is desired, filaments of nonbiodegradable materials may be used. Non-limiting examples of nonbiodegradable materials include silk, polyesters (e.g., polyester terephthalate, dacron, etc.), polyamides (e.g., nylons), polyethylene, polypropylene, cellulose, polystyrene, polyacrylates, polyvinyls, polytetrafluoroethylenes (PTFE), expanded PTFE (ePTFE), and polyvinylidine fluoride. Other polymers will be apparent to the skilled artisan.

In other embodiments, the three dimensional scaffold or framework is a combination of different biodegradable filaments or combinations of biodegradable and non-biodegradable materials. A non-biodegradable material provides stability to the structures during culturing and increases the tensile strength when used as a suture material. The biodegradable material may be coated onto the non-biodegradable material or woven, braided or formed into a mesh. For instance, a sheath may be made of biodegradable filaments while the core is made of nonbiodegradable filaments. Various combinations of biodegradable and non-biodegradable materials may be used.

The three dimensional framework may be braided into a cord, such as a suture, by techniques conventional in the art. Processes and methods for producing braided or knitted tubular sheaths, including various types of sutures, are described in, e.g., in U.S. Pat. Nos. 3,773,919; 3,792,010; 3,797,499; 3,839,297; 3,867,190; 3,878,284; 3,982,543; 4,047,533; 4,060,089; 4,137,921; 4,157,437; 4,234,775; 4,237,920; 4,300,565; 4,523,591; 5,019,093, 5,059,213; 5,133,738; 5,181,923; 5,261,886; 5,306,289; 5,314,446; 5,456,697; 5,662,682; 6,045,071; 6,164,339; and 6,184,499. All publications are incorporated herein by reference. An exemplary method for forming filaments, such as PLGA, is a melt spinning process. Biocompatible bioabsorbable multifilament sutures are also available commercially under such tradenames as Dexon®, Vicryl®, and Polysorb® from various suppliers, such as Ethicon, Inc. (Somerville, N.J., USA), United States Surgical (Norwalk, Conn., USA), and Prodesco (Perkasie, Pa., USA)

Cords and braided sutures may be subjected to further processing, such as hot stretching, scouring, annealing, coating, tipping, cutting, needle attachment, packaging and sterilization prior to inoculation with the cells. To alter its mechanical characteristics, the filament can be stretched to reorient the molecule chains in the polymer. Annealing can be carried out to fix the characteristics of the filament, such as to maintain the polymer orientation, alter tensile strength, and fix geometric stability of the filaments.

The cord or braid may be of various axial diameters or dimensions depending on the desired application. Braided or woven frameworks may have smaller diameters when used as sutures for holding tissues together while larger diameters may be used when administered into tissues or organs for repair of tissue damage. In various embodiments, the diameters of the braided or woven frameworks range are about 0.05 mm, about 0.10 mm, about 0.2 mm, about 0.5 mm, about 1 mm, about 1.5 mm, or about 2 mm. It is to be understood that the diameters may be smaller or larger depending on the clinical application, the desired tensile strength, and the amount of cells attached to the framework.

### 5.2 Cells and Culture Conditions

For forming the three dimensional tissues, the biocompatible materials forming the scaffolds are inoculated with the appropriate cells and grown under suitable conditions to promote formation of a three dimensional tissue and promote production of a conditioned medium with the hair growth promoting properties. Cells can be obtained directly from a donor, from cell cultures made from a donor, or from established cell culture lines. In some embodiments, cells can be obtained in quantity from any appropriate cadaver organ or fetal sources. In some embodiments, cells of the same species, and optionally the same or similar immunohistocompatibility profile, may be obtained by biopsy, either from the subject or a close relative, which are then grown to confluence in culture using standard conditions and used as needed. The characterization of the donor cells with respect to the immunohistocompatibility profile are made in reference to the subject being administered the compositions.

Accordingly, in some embodiments, the cells are autologous. Because the three dimensional tissues derive from recipient's own cells, the possibility of an immunological reaction against the administered cells and/or products produced by the cells may be minimized. In some embodiments, the cells may be initially cultured on two-dimensional surfaces typically used in cell culture (e.g., plates) prior to seeding the three dimensional framework.

In other embodiments, the cells are obtained from a donor who is not the intended recipient of the compositions. The relation of the donor to the recipient is defined by similarity or identity of the multihistocompatibility complex (MHC). In some embodiments, the donor cells are syngeneic cells in that the cells derive from a subject who is genetically identical at the MHC to the intended recipient. In other embodiments, the cells are allogeneic cells in that the cells derive from a subject who is of the same species as the intended recipient but whose MHC complex is different. Where the cells are allogeneic, the cells may be from a single donor or comprise a mixture of cells from different donors who themselves are allogeneic to each other. In further embodiments, the cells are xenogenic cells in that the cells are derived from a species different than the intended recipient.

In various embodiments, the cells inoculated onto the framework can be stromal cells comprising fibroblasts, with or without other cells, as further described below. In some embodiments, the cells are stromal cells that are typically derived from connective tissue, including, but not limited to: (1) bone; (2) loose connective tissue, including collagen and elastin; (3) the fibrous connective tissue that forms ligaments and tendons, (4) cartilage; (5) the extracellular matrix of blood; (6) adipose tissue, which comprises adipocytes; and (7) fibroblasts.

Stromal cells can be derived from various tissues or organs, such as skin, heart, blood vessels, bone marrow, skeletal muscle, liver, pancreas, brain, foreskin, which can be obtained by biopsy (where appropriate) or upon autopsy.

In some embodiments, the cells comprise fibroblasts, which can be from a fetal, neonatal, adult origin, or a combination thereof. In some embodiments, the stromal cells comprise fetal fibroblasts, which can support the growth of a variety of different cells and/or tissues. As used herein, a fetal fibroblast refers to fibroblasts derived from fetal sources. As used herein, neonatal fibroblast refers to fibroblasts derived from newborn sources. Under appropriate conditions, fibroblasts can give rise to other cells, such as bone cells, fat cells, and smooth muscle cells and other cells of mesodermal origin. In some embodiments, the fibroblasts comprise dermal fibroblasts, which are fibroblasts derived from skin. Normal human dermal fibroblasts can be isolated from neonatal foreskin. These cells are typically cryopreserved at the end of the primary culture.

In other embodiments, the three-dimensional tissue can be made using stem or progenitor cells, either alone, or in combination with any of the cell types discussed herein. Exemplary stern and progenitor cells include, by way of example and not limitation, embryonic stem cells, hematopoietic stem cells, neuronal stem cells, epidermal stem cells, and mesenchymal stem cells.

In some embodiments, a “specific” three-dimensional tissue can be prepared by inoculating the three-dimensional scaffold with cells derived from a particular organ, i.e., skin, heart, and/or from a particular individual who is later to receive the cells and/or tissues grown in culture in accordance with the methods described herein.

As discussed above, additional cells may be present in the culture with the stromal cells. These additional cells may have a number of beneficial effects, including, among others, supporting long term growth in culture, enhancing synthesis of growth factors, and promoting attachment of cells to the three dimensional scaffold. Additional cell types include as non-limiting examples, smooth muscle cells, cardiac muscle cells, endothelial cells, skeletal muscle cells, endothelial cells, pericytes, macrophages, monocytes, and adipocytes. Such cells may be inoculated onto the three-dimensional framework along with fibroblasts, or in some embodiments, in the absence of fibroblasts. These stromal cells may be derived from appropriate tissues or organs, including, by way of example and not limitation, skin, heart, blood vessels, bone marrow, skeletal muscle, liver, pancreas, and brain. In other embodiments, one or more other cell types, excluding fibroblasts, are inoculated onto the three-dimensional scaffold. In still other embodiments, the three-dimensional scaffolds are inoculated only with fibroblast cells.

Cells such as stromal cells may be isolated by disaggregating an appropriate organ or tissue using techniques known to those skilled in the art. For example, the tissue or organ can be disaggregated mechanically and/or treated with digestive enzymes and/or chelating agents that weaken the connections between neighboring cells and thereby disperse the tissue into a suspension of individual cells without appreciable cell breakage. Enzymatic dissociation can be accomplished by mincing the tissue and treating the minced tissue with any of a number of digestive enzymes either alone or in combination. Non-limiting examples of enzymes include, among others, trypsin, chymotrypsin, collagenase, elastase, and/or hyaluronidase. DNase, pronase, and dispase. Mechanical disruption can also be accomplished by a number of methods including, but not limited to, the use of grinders, blenders, sieves, homogenizers, pressure cells, or insonators. For a review of tissue disaggregation techniques, see Freshney, Culture of Animal Cells. A Manual of Basic Technique, 2d Ed., A. R. Liss, Inc., New York, 1987, Ch. 9, pp. 107-126.

Suspensions of individual cells can be fractionated into subpopulations from which the fibroblasts and/or other stromal cells and/or elements can be obtained. Standard techniques for cell separation and isolation include, by way of example and not limitation, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis and fluorescence-activated cell sorting. For a review of clonal selection and cell separation techniques, see Freshney, supra, Ch. 11 and 12, pp. 137-168.

After inoculation of the three dimensional scaffolds, the cell culture is incubated in an appropriate nutrient medium and incubation conditions that supports growth of cells into the three dimensional tissues. Many commercially available media such as Dulbecco's Modified Eagles Medium (DMEM), RPMI 1640, Fisher's, Iscove's, and McCoy's, may be suitable for supporting the growth of the cell cultures. The medium may be supplemented with additional salts, carbon sources, amino acids, serum and serum components, vitamins, minerals, reducing agents, buffering agents, lipids, nucleosides, antibiotics, attachment factors, and growth factors. Formulations for different types of culture media are described in various reference works available to the skilled artisan (e.g. Methods for Preparation of Media, Supplements and Substrates for Serum Free Animal Cell Cultures, Alan R. Liss, New York (1984); Tissue Culture: Laboratory Procedures, John Wiley & Sons, Chichester, England (1996); Culture of Animal Cells, A Manual of Basic Techniques, 4^{th} Ed., Wiley-Liss (2000). Incubation conditions will be under appropriate conditions of pH, temperature, and gas (e.g. O₂, CO₂, etc) that support growth of cells. In some embodiments, the three-dimensional cell culture can be suspended in the medium during the incubation period in order to maximize proliferative activity and generate factors that facilitate the desired biological activities of the conditioned media. In addition, the culture may be “fed” periodically to remove the spent media, depopulate released cells, and add new nutrient source. During the incubation period, the cultured cells grow linearly along and envelop the filaments of the three-dimensional scaffold before beginning to grow into the openings of the scaffold.

The three dimensional tissues described herein have extracellular matrix that is present on the scaffold or framework. In some embodiments, the extracellular matrix comprises various collagen types, different proportions of which can affect the growth of the cells that come in contact with the three dimensional tissues. The proportions of extracellular matrix (ECM) proteins deposited can be manipulated or enhanced by selecting fibroblasts which elaborate the appropriate collagen type. This can be accomplished in some embodiments using monoclonal antibodies of an appropriate isotype or subclass that are capable of activating complement and which define particular collagen types. In other embodiments, solid substrates, such as magnetic beads, may be used to select or eliminate cells that have bound antibody. Combination of these antibodies can be used to select (positively or negatively) the fibroblasts which express the desired collagen type. Alternatively, the stroma used to inoculate the framework can be a mixture of cells which synthesize the desired collagen types. The distribution and origins of the exemplary type of collagen are shown in Table 1.

TABLE I
Distributions and Origins of Various Types of Collagen

| Collagen | | |
|---|---|---|
| Type | Principle Tissue Distribution | Cells of Origin |
| I | Loose and dense ordinary | Fibroblasts and reticular |
| | connective tissue; collagen fibers | cells; smooth muscle |
| | | cells |
| | Fibrocartilage | |
| | Bone | Osteoblast |
| | Dentin | Odontoblasts |
| II | Hyaline and elastic cartilage | Chondrocytes |
| | Vitreous body of the eye | Retinal cells |
| III | Loose connective tissue; reticular | Fibroblasts and reticular |
| | fibers | cells |
| | Papillary layer of dermis | |
| | Blood vessels | Smooth muscle cells; |
| | | endothelial cells |
| IV | Basement membranes | Epithelial and endothelial |
| | | cells |
| | Lens capsule of the eye | Lens fiber |
| V | Fetal membranes; placenta | Fibroblasts |
| | Basement membranes | |
| | Bone | |
| | Smooth muscle | Smooth muscle cells |
| VI | Connective tissue | Fibroblasts |
| VII | Epithelial basement membranes; | Fibroblasts; keratinocytes |
| | anchoring fibrils | |
| VIII | Cornea | Corneal fibroblasts |
| IX | Cartilage | |
| X | Hypertrophic cartilage | |
| XI | Cartilage | |
| XII | Papillary dermis | Fibroblasts |
| XIV | Reticular dermis | Fibroblasts |
| (undulin) | | |
| XVII | P170 bullous pemphigoid antigen | Keratinocytes |

During culturing of the three-dimensional tissues, proliferating cells may be released from the framework and stick to the walls of the culture vessel where they may continue to proliferate and form a confluent monolayer. To minimize this occurrence, which may affect the growth of cells, released cells may be removed during feeding or by transferring the three-dimensional cell culture to a new culture vessel. Removal of the confluent monolayer or transfer of the cultured tissue to fresh media in a new vessel maintains or restores proliferative activity of the three-dimensional cultures. In some embodiments, removal or transfers may be done in a culture vessel which has a monolayer of cultured cells exceeding 25% con fluency. Alternatively, the culture in some embodiments is agitated to prevent the released cells from sticking; in others, fresh media is infused continuously through the system. In some embodiments, two or more cell types can be cultured together either at the same time or one first followed by the second (e.g., fibroblasts and smooth muscle cells or endothelial cells).

In some embodiments, the three dimensional tissue may be prepared in bioreactors, such as those described in U.S. Pat. Nos. 5,763,267; 5,827,729; 6,008,049; 6,060,306; 6,121,042; and 6,218,182, the disclosures of which are incorporated herein by reference. Impellers in the bioreactors may be modified to limit attachment of the three dimensional tissues to the hubs. In addition, the working volume of impellers may be reduced by shortening the impeller shafts, thereby providing flexibility in culturing the three dimensional tissues.

In various embodiments, the three dimensional tissues may be defined by a characteristic set, fingerprint, repertoire, or suite of cellular products produced by the cells, such as growth factors. In the three dimensional tissues specifically exemplified herein, the cell cultures are characterized by expression and/or secretion of the factors given in Table II

TABLE II
Three Dimensional Tissue Expressed Factors

| | | Secreted Amount |
|---|---|---|
| Growth Factor | Expressed by Q-RT-PCR | Determined by ELISA |
| VEGF | 8 × 10⁶ copies/ug RNA | 700 pg/10⁶ cells/day |
| PDGF A chain | 6 × 10⁵ copies/ug RNA | |
| PDGF B chain | 0 | 0 |
| IGF-1 | 5 × 10⁵ copies/ugRNA | |
| EGF | 3 × 10³ copies/ug RNA | |
| HBEGF | 2 × 10⁴ copies/ug RNA | |
| KGF | 7 × 10⁴ copies/ug RNA | |
| TGF-β1 | 6 × 10⁶ copies/ug RNA | 300 pg/10⁶ cells/day |
| TGF-β3 | 1 × 10⁴ copies/ug RNA | |
| HGF | 2 × 10⁴ copies/ug RNA | 1 ng/10⁶ cells/day |
| IL-1a | 1 × 10⁴ copies/ug RNA | Below detection |
| IL-1b | 0 | |
| TNF-a | 1 × 10⁷ copies/ug RNA | |
| TNF-b | 0 | |
| IL-6 | 7 × 10⁶ copies/ug RNA | 500 pg/10⁶ cells/day |
| IL-8 | 1 × 10⁷ copies/ug RNA | 25 ng/10⁶ cells/day |
| IL-12 | 0 | |
| IL-15 | 0 | |
| NGF | 0 | |
| G-CSF | 1 × 10⁴ copies/ug RNA | 300 pg/10⁶ cells/day |
| Angiopoietin | 1 × 10⁴ copies/ug RNA | |

In addition to the above list of growth factors, the three dimensional tissue is also characterized by the expression of Wnt proteins, wherein the Wnt proteins comprise at least Wnt5a, Wnt7a, and Wnt11. Descriptions of these specific Wnt proteins are given below.

It is to be understood that additional cell products, including other growth factors, may be produced by the cell cultures such that the scope of the three dimensional tissue and the conditioned media produced therefrom are not to be limited by the descriptions above.

### 5.3 Genetically Engineered Cells

Genetically engineered three-dimensional stromal tissue may be prepared as described in U.S. Pat. No. 5,785,964 which is incorporated herein by reference. Generally, a genetically-engineered stromal tissue may serve as a gene delivery vehicle for sustained release of growth factors. Cells may be engineered to express an exogenous gene product. In some embodiments, stromal cells that can be genetically engineered include, by way of example and not limitation, fibroblasts, smooth muscle cells, cardiac muscle cells, mesenchymal stem cells, and other cells found in loose connective tissue such as endothelial cells, macrophages, monocytes, adipocytes, pericytes, and reticular cells found in bone marrow.

The cells and tissues may be engineered to express a target gene product which may impart a wide variety of functions, including, but not limited to, promote proliferation of cells in culture, enhance production of growth factors promoting hair growth, enhance production of factors promoting vascularization, and produce factors that counteract the effect of compounds that impair hair growth. The target gene product may be a peptide or protein, such as an enzyme, hormone; cytokine; regulatory protein, such as a transcription factor or DNA binding protein; structural protein, such as a cell surface protein; or the target gene product may be a nucleic acid such as a ribosome or antisense molecule. In a preferred embodiment, the target gene product is one or more Wnt proteins, which play a role in differentiation and proliferation of a variety of cells as described below (see, e.g., Miller, J. R., 2001, Genome Biology 3:3001.1-3001.15).

In some embodiments, the target gene products which provide enhanced properties to the genetically engineered cells, include but are not limited to, gene products which enhance cell growth. Non-limiting examples of such vascular endothelial growth factor (VEGF), hepatocyte growth factor (HGF), fibroblast growth factors (FGF), platelet derived growth factor (PDGF), epidermal growth factor (EGF), transforming growth factor (TGF), and Wnt factors. Where the recombinantly engineered cells are made to express Wnt factors, specific Wnt factors for expression in the cell include, among others, one or more of Wnt5a, Wnt7a, and Wnt11. In other embodiments, the cells and tissues are genetically engineered to express target gene products which result in cell immortalization, e.g. oncogenes or telomerese.

In other embodiments, the cells and tissues are genetically engineered to express gene products which provide protective functions in vitro such as cyropreservation and anti-desiccation properties, e.g., trehalose (U.S. Pat. Nos. 4,891,319; 5,290,765; and 5,693,788). The cells and tissues of the present invention may also be engineered to express gene products which may provide a protective function in vivo, such as those which would protect the cells from an inflammatory response and protect against rejection by the host's immune system, such as HLA epitopes, major histocompatibility epitopes, immunoglobulin and receptor epitopes, epitopes of cellular adhesion molecules, cytokines, and chemokines.

There are a number of ways that the target gene products may be engineered to be expressed by the cells and tissues of the present invention. The target gene products may be engineered to be expressed constitutively or in a tissue-specific or stimuli-specific manner. In accordance with this aspect of the invention, the nucleotide sequences encoding the target gene products may be operably linked to promoter elements which are constitutively active, tissue-specific or induced upon presence of a specific stimulus.

In various embodiments, the nucleotide sequences encoding the target gene products are operably linked to regulatory promoter elements that are responsive to shear or radial stress. In this instance, the promoter element would be turned on by passing blood flow (shear) as well as the radial stress that is induced as a result of the pulsatile flow of blood through the heart or vessel.

Examples of other regulatory promoter elements include tetracycline responsive elements, nicotine responsive elements, insulin responsive element, glucose responsive elements, interferon responsive elements, glucocorticoid responsive elements estrogen/progesterone responsive elements, retinoid acid responsive elements, viral transactivators, early or late promoter of SV40 adenovirus, the lac system, the trp system, the TAC system, the TRC system, the promoter for 3-phosphoglycerate and the promoters of acid phosphatase. In addition, artificial response elements could be constructed, composed of multimers of transcription factor binding sites and hormone-response elements similar to the molecular architecture of naturally-occurring promoters and enhancers (see, e.g. Herr and Clarke, 1986, J Cell 45(3): 461-70). Such artificial composite regulatory regions could be designed to respond to any desirable signal and be expressed in particular cell-types depending on the promoter/enhancer binding sites selected. Techniques for constructing the expression systems and genetically engineering cells are found in various reference works, such as Sambrook et al., 2000, Molecular Cloning: A Laboratory Manual, 3^{rd} Ed., Cold Spring Harbor Press, Cold Spring Harbor, N.Y.; Current Protocols in Molecular Biology, Ausubel et al., eds., John Wiley & Sons, 1988, updates to 2005; and Current Protocols in Cell Biology. Bonifacino et al. eds., John Wiley & Sons, 2001, updates to 2005. All publications incorporated herein by reference.

### 5.4 Use of Wnt Factors Produced from Three Dimensional Tissue to Promote Hair Growth

The three dimensional tissues herein produce Wnt factors, which may play various roles in hair follicle development. Wnt is a signaling molecule having roles in a myriad of cellular pathways and cell-cell interaction processes. Wnt signaling has been implicated in tumorigenesis, early mesodermal patterning of the embryo, morphogenesis of the brain and kidneys, regulation of mammary gland proliferation, and Alzheimer's disease.

“Wnt” or “Wnt protein” as used herein refers to a protein with one or more of the following functional activities: (1) binding to Wnt receptors, also referred to as Frizzled proteins, (2) modulating phosphorylation of Dishevelled protein and cellular localization of Axin protein (3) modulation of cellular β-catenin levels and corresponding signaling pathway, (4) modulation of TCF/LEF transcription factors, and (5) increasing intracellular calcium and activation of Cat⁺² sensitive proteins (e.g., calmodulin dependent kinase). “Modulation” as used in the context of Wnt proteins refers to an increase or decrease in cellular levels, changes in intracellular distribution, and/or changes in functional (e.g., enzymatic) activity of the molecule modulated by Wnt.

“Wnt mediated signaling” refers to activation of a cellular signaling pathway initiated by or dependent on interaction of Wnt protein and its cognate receptor protein. As a point of reference, the canonical Wnt signaling pathway involves binding of the Wnt protein to its corresponding cellular receptor, the Frizzled proteins. Receptor activation tranduces a signal by phosphorylation of the protein Dishevelled, which interacts with Axin. This interaction disrupts the formation of a cellular complex comprised of the proteins Axin, Adenomatous Polyposis Coli (APC), and glycogen synthase kinase-3β (GSK-3) that is believed to regulate β-catenin activity by promoting its degradation via a proteosome mediated pathway. Wnt signaling, through its action on Dishevelled and Axin, inhibits degradation of β-catenin, thereby leading to (β-catenin accumulation in the cytoplasm and nucleus. β-catenin then interacts with the transcription factor TCF/LEF and promotes its translocation into the nucleus, where the protein complex modulates the transcription of various target genes.

It is to be understood, however, that Wnt signaling is not restricted to the canonical pathway, and that cells may have alternative pathways affected by signal transduction mediated by Wnt. β-catenin has been shown to interact with other types of transcription factors, such as p300/CBP, BRG-1, and LIM domain protein FHL-2. In addition, several non-canonical Wnt signaling pathways have been elucidated that act independently of β-catenin (see, e.g., Lustig and Behrens, 2003, J. Cancer Res. Clin. Oncol. 129:199-221; Polakis, P., 2000, Genes Dev. 14:1837-1851). In one noncannonical pathway, Wnt binds to the Frizzled receptor resulting in the activation of heterotrimeric G-proteins and subsequent mobilization of phospholipase C and phosphodiesterase. This activation results in a decrease in cGMP levels, an increase in intracellular Ca⁺², and activation of protein kinase C and other Ca⁺² regulated proteins. A second non-canonical pathway is the planar cell polarity (PCP) pathway that defines polarity in select epithelial tissues, particularly along an axis perpendicular to the apical-basal border. In vertebrates, it may contribute to the differentiation and orientation of inner ear hair cell stereocilia and direct the expansion of mesoderm and neuroectoderm during gastrulation (Dabdoub and Kelley, 2005, J. Neurobiol. 64(4):446-57). It is thought that activation of the PCP pathway occurs by Wnt binding to Frizzled, which activates Dishevelled. Dishevelled then recruits RhoA/Rac, which ultimately leads to JNK (c-jun NH2-terminal kinase) pathway activation. A major target of the JNK pathway appears to be the AP-1 (activator protein-1) transcription factor.

“Wnt” or “Wnt proteins” are also characterized structurally by their sequence similarity or identity to mouse Wnt-1 and Wingless in Drosophila. As used herein “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e. gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols in Molecular Biology, John Wiley & Sons, Inc., 1995 Supplement). Examples of algorithms that are suitable for determining percent sequence identity and sequence similarity are the BLAST and BLAST 2.0 algorithms, which are described in Altschul et al., 1990, J. Mol. Biol. 215:403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

While all of the above mentioned algorithms and programs are suitable for a determination of sequence alignment and % sequence identity, for determination of % sequence identity in some embodiments the BESTFIT or GAP programs in the GCG Wisconsin Software package (Accelrys, Madison Wis.), is used with the default parameters provided.

Of relevance to the present disclosure are Wnt proteins expressed in mammals, such as rodents, felines, canines, ungulates, and primates. For instance, human Wnt proteins that have been identified share 27% to 83% amino-acid sequence identity. Additional structural characteristics of Wnt protein are a conserved pattern of about 23 or 24 cysteine residues, a hydrophobic signal sequence, and a conserved asparagine linked oligosaccharide modification sequence. Some Wnt proteins are also lipid modified, such as with a palmitoyl group (Wilkert et al., 2003, Nature 423(6938):448-52). Exemplary Wnt proteins and its corresponding genes expressed in mammals include, among others, Wnt 1, Wnt 2. Wnt 2B, Wnt 3, Wnt3A, Wnt4, Wnt 4B, Wnt5A, Wnt 5B, Wnt 6, Wnt 7A, Wnt 7B, Wnt8A, Wnt8B, Wnt9A, Wnt9B, Wnt10A, Wnt11, and Wnt 16. Other identified forms of Wnt, such as Wnt12, Wnt13. Wnt14, and Wnt 15, appear to fall within the proteins described for Wnt 1-11 and 16. Protein and amino acid sequences of each of the mammalian Wnt proteins are available in databases such as SwissPro and Genbank (NCBI) (see, e.g., U.S. Published Application No. 20040248803, incorporated herein by reference). Within the scope of “Wnt” and “Wnt proteins” are protein fragments, variants, and mutants of the identified Wnt proteins, where the fragments, variants, and mutants have the functional activities characteristic of the family of Wnt proteins.

In the embodiments herein, the “suite”, “repertoire”, “signature” or “fingerprint” of Wnt factors elaborated by the three dimensional tissues may be used to modulate hair growth. Wnt factors produced by the three dimensional tissues comprise at least Wnt5a, Wnt7a, and Wnt11, which defines a characteristic or signature of the Wnt proteins present in the conditioned media. As used herein, Wnt5a refers to a Wnt protein with the functional activities described above and sequence similarity to human Wnt protein with the amino acid sequence in NCBI Accession Nos. AAH74783 (gI:50959709) or AAA 16842 (gI:348918) (see also, Danielson et al., 1995, J. Biol. Chem. 270(52):31225-34). Wnt7a refers to a Wnt protein with the functional properties of the Wnt proteins described above and sequence similarity to human Wnt protein with the amino acid sequence in NCBI Accession Nos. BAA82509 (gI:5509901); AAC51319.1 (GI:2105100); and O00755 (gI:2501663) (see also, Ikegawa et al., 1996, Cytogenet Cell Genet. 74(1-2):149-52; Bui et al., 1997, Gene 189(1):25-9). Wnt11 refers to a Wnt protein with the functional activities described above and sequence similarity to human Wnt protein with the amino acid sequence in NCBI Accession Nos. BAB72099 (gI:17026012); CAA74159 (gI:3850708); and CAA73223.1 (gI:3850706) (see also, Kirikoshi et al., 2001, Int. J. Mol. Med. 8(6):651-6); Lako et al., 1998, Gene 219(1-2): 101-10). As used herein in the context the specific Wnt proteins, “sequence similarity” refers to an amino acid sequence identity of at least about 80% or more, at least about 90% or more, at least about 95% or more, or at least about 98% or more when compared to the reference sequence. For instance, human Wnt7a displays about 97% amino acid sequence identity to murine Wnt7a while the amino acid sequence of human Wnt7a displays about 64% amino acid identity to human Wnt5a (Bui et al., supra).

In other embodiments, isolated Wnt proteins are used alone to modulate hair growth or as supplement to the conditioned media produced from the three dimensional tissues. As noted above, a number of different Wnt proteins have been determined to be produced in the three dimensional tissues and may be isolated by the methods described herein. Isolated Wnt proteins that may be useful for the methods herein include Wnt5, Wnt7 and Wnt is 11a, as described above.

The suite of Wnt proteins elaborated by the cell culture or the individual Wnt proteins may be isolated by various techniques available to the skilled artisan. Because of the lipid modification of Wnt proteins, purification typically uses detergents to solubilize and maintain the activity of Wnt proteins. These methods are described in Willert et al., 2003, Nature 423(6938):448-52 and U.S. Published Application No. 20040248803, incorporated herein by reference. The Wnt proteins made in the three dimensional tissue may be solubilized with non-anionic detergents or zwitterionic detergents at a concentration of from about 0.25% to about 2.5%, at a concentration of from about 0.5% to 1.5%, or at a concentration of about 1%. In some embodiments, suitable non-anionic detergents for solubilizing the Wnts are members of detergents available under the tradename Triton, including Triton X-15, Triton X-35, Triton X45, Triton X-100, Triton X-102, Triton X-114, and Triton X-165. In some embodiments, solubilization may be combined with other purification techniques to obtained isolated or enriched preparations of Wnt. These include other art known techniques such as reverse phase chromatography high performance liquid chromatography, ion exchange chromatography, gel electrophoresis, affinity chromatography (e.g., dye ligand with Cibaron Blue) of solubilized Wnt proteins. The actual conditions used to isolate the Wnt proteins will depend, in part, on factors such as net charge, hydrophobicity, hydrophilicity, molecular weight, etc., and will be apparent to those having skill in the art, as described in U.S. Published Application No. 20040248803.

In other embodiments, antibodies to identified Wnt proteins may be used en mass to isolate the suite of Wnt proteins produced by the three dimensional tissues. In other embodiments, an antibody directed to a common epitope expressed in different Wnt proteins may be used to isolate multiple Wnt proteins. In still other embodiments, antibodies to specific Wnt proteins (e.g., Wnt5a, Wnt7a, and Wnt11) may be used to isolate a single type of Wnt protein produced by the cultures. Antibodies may be immobilized in a column or solid surface (e.g., magnetic beads, agarose beads, etc.) to isolate the Wnt proteins or alternatively precipitated by agents such as Staph A protein or other antibody binding agents. Procedures for antibody based purification are described in many reference works, such as Ausubel, Current Methods in Molecular Biology, John Wiley & Sons, updates to 2005; Harlow and Lane, 1988, Antibodies: A Laboratory Manual, Cold Spring Harbor Laboratory Press, Cold Spring Harbor, N.Y.; Scopes, 1984, Protein Purification: Principles and Practice, Springer Verlag New York, Inc., N.Y.; and Livingstone, 1974, Methods In Enzymology: Immunoaffinity Chromatography of Proteins 34:723 731. All publications incorporated herein by reference.

In other embodiments, the Wnt proteins may be made by recombinant methods using methods well known in the art, for example, as described in U.S. Published Application No. 20040248803.

### 5.5 Assays for Hair Growth

The conditioned medium and the Wnt factors prepared from the three dimensional tissue may be assayed in various ways, including in vitro systems, animal models, and subjects afflicted with hair loss. In vitro assays include whole skin explants and dissociated hair follicle cells. Whole skin explants from human and mouse sources are described in Li et al., 1992, Proc. Natl. Acad. Sci. USA 89:8764-8768; Li et al., 1992, Cell Dev. Biol. 28:695-698; Paus et al., 1994, J. Dermatol. Sci. 7:202-209; Paus et al., 1988, and Yale Biol. Med. 61:467-476. These skin culture systems permit hair follicle development, anagen I-VI development, and follicle pigmentation, thus serving as suitable systems for examining the effect of growth factors and conditioned media (Botchkarev et al., 1998, J. Invest. Dermatol. 111:279-285; Botchharev et al., 1999, J. Invest. Dermatol. 113:425-427; Foitzik et al., 1999, Devel. Biol. 212:278-289; and St. Jacques et al., 1998, Curr. Biol. 8:1058-1068).

Other in vitro systems for measuring the effect of conditioned media use dissected hair follicle cells, isolated mesenchymal cells, or hair bulb keratinocytes from the dermal layer of skin and cultured on coated plates (e.g., Tanigaki et al., 1990, Arch. Dermatol. Res. 282:402-407; Jahoda et al., 1984. Nature 311:560-562; Jahoda et al., 1981, Br. J. Dermatol. 105:623-627; Messenger et al., 1984, Br. J. Dermatol. 110:685-689; Warren et al., 1992. J. Invest. Dermatol. 98:693-699). Cell growth and differentiation of mesenchymal and dermal papillar cells provide an indication of effect of the conditioned medium on development of the hair follicle. Determining growth and differentiation is typically done by vital or cell specific stains and detecting expression of differentiation markers (e.g., by antibodies or gene expression profiles).

Various ex vivo assays combine in vitro and in vivo approaches. In one type of assay system, the hair follicles are removed, grown in culture, and then transplanted into the skin of immunodeficient animals. These reconstituted systems as well as ex vivo organ cultures are described in Lichti et al., 1993, J. Invest. Dermatol. 101:124S-129S; Rogers et al., 1987. J. Invest. Dermatol. 89:369-379; Weinberg et al., 1993, J. Invest. Dermatol. 100:229-236; Kamimura et al., 1997, J. Invest. Dermatol. 109:534-540; Kishimoto et al., 1999, Proc. Natl. Acad. Sci. USA 96:7336-7341; Moscona. A., 1961. Exp. Cell Res. 22:455-475; Takeda et al., 1996, “Reconstitution of hair follicles by rotation culture,” In Hair Research for Next Millenium, van Neste, D. and Randall, V. A. eds., Elsevier, Amsterdam, p 191-193; and Kobayashi et al., 1989, J. Invest. Dermatol. 92:278-282. All publications incorporated herein by reference.

In vivo assays for hair growth typically involve shaving off the hair of a suitable animal such as a mouse, rat, sheep or any other hairy mammal and determining hair regrowth following administration of the test material on the shaved region. An in vivo animal test system similar to humans is the macaque, which also displays forms of hereditary alopecia (Uno, W. P., 1991, Ann NY Acad. Sci. 642:107-124). Typical assays, however, use pigmented animals in which the truncal pigmentation is dependent on activity of follicular melanocytes, for example C57BL/6 and C3H mice. Because pigment production occurs only during the anagen phase in these animals, hair regrowth is easily assessed by evaluating skin color. Removal of the hair may be carried out when the hair follicles are in a specified phase of hair growth, such as the telogen phase (e.g., Takahashi et al., 1998, J. Invest. Dermatol. 112:310-316). Quantitative measurements of hair regrowth are assessed by photographing the shaved area and evaluating pigmentation levels and/or by measuring length and density of hair follicle in the test region.

As will be apparent to the skilled artisan, other methods for assessing growth and differentiation of cells responsible for hair growth may be used for the purposes defined herein and is not restricted to the various embodiments presented in this disclosure.

### 5.6 Processing of Conditioned Media and Three Dimensional Tissues, and Pharmaceutical Compositions Thereof

In various embodiments, conditioned media produced by the three dimensional tissues may be used directly or processed in various ways. The medium may be subject to lyophilization for preserving and/or concentrating the factors that promote hair growth. Various biocompatible preservatives, cryoprotectives, and stabilizer agents may be used to preserve activity where required. Non-limiting examples of biocompatible agents include, among others, glycerol, dimethyl sulfoxide, and trehalose. The lyophilizate may also have one or more excipients such as buffers, bulking agents, and tonicity modifiers. The freeze-dried media may be reconstituted by addition of a suitable solution or pharmaceutical diluent, as further described below.

In some embodiments, the conditioned media may be processed by precipitating the active components (e.g., growth factors) in the media. Precipitation may use various procedures, such as salting out with ammonium sulfate or use of hydrophilic polymers, for example polyethylene glycol.

In other embodiments, the conditioned media is subject to filtration using various selective filters. Processing the conditioned media by filtering is useful in concentrating the factors that promote growth of hair and also removing small molecules and solutes used in the culture medium. Filters with selectivity for specified molecular weights include <5000 daltons, <10,000 daltons, and <15,000 daltons. Other filters may be used and the processed media assayed for hair growth promoting activity as described herein. Exemplary filters and concentrator system include those based on, among others, hollow fiber filters, filter disks, and filter probes (see, e.g., Amicon Stirred Ultrafiltration Cells).

In still other embodiments, the conditioned medium is subject to chromatography to remove salts, impurities, or fractionate various components of the medium. Various chromatographic techniques may be employed, such as molecular sieving, ion exchange, reverse phase, and affinity chromatographic techniques. For processing conditioned medium without significant loss of bioactivity, mild chromatographic media may be used. Non-limiting examples include, among others, dextran, agarose, polyacrylamide based separation media (e.g., available under various tradenames, such as Sephadex, Sepharose, and Sephacryl).

The conditioned medium may be used directly without additional additives, or prepared as pharmaceutical compositions with various pharmaceutically acceptable excipients, vehicles or carriers. A “pharmaceutical composition” refers to a form of the conditioned media and at least one pharmaceutically acceptable vehicle, carrier, or excipient. For intradermal, subcutaneous or intramuscular administration, the compositions may be prepared in sterile suspension, solutions or emulsions of the conditioned media in aqueous or oily vehicles. The compositions may also contain formulating agents, such as suspending, stabilizing or dispersing agents. Formulations for injection may be presented in unit dosage form, ampules in multidose containers, with or without preservatives. Alternatively, the compositions may be presented in powder form for reconstitution with a suitable vehicle including, by way of example and not limitation, sterile pyrogen free water, saline, buffer, or dextrose solution.

In still other embodiments, the conditioned media is formulated as liposomes. The growth factors may be introduced or encapsulated into the lumen of liposomes for delivery and for extending life time of the active factors. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids; and other lipids (see, e.g., U.S. Pat. No. 5,833,948).

Cationic lipids are also suitable for forming liposomes. Generally, the cationic lipids have a net positive charge and have a lipophilic portion, such as a sterol or an acyl or diacyl side chain. In some embodiments, the head group is positively charged. Typical cationic lipids include 1,2-dioleyloxy-3-(trimethylamino) propane; N-[1-(2,3,-ditetradecycloxy)propyl]-N,N-dimethyl-N—N-hydroxyethylammonium bromide; N-[1-(2,3-dioleyloxy)propyl]-N,N-dimethyl-N-hydroxy ethylammonium bromide; N-[1-(2,3-dioleyloxy)propyl]-N,N,N-trimethylammonium chloride; 3-[N—(N′,N′-dimethylaminoethane) carbamoyl]choiesterol; and dimethyldioctadecylammonium.

In other embodiments, the liposomes comprise fusogenic liposomes, which are characterized by their ability to fuse with a cell membrane upon appropriate change in physiological condition or by presence of fusogenic component, particularly a fusogenic peptide or protein. In some embodiments, the fusogenic liposomes are pH and temperature sensitive in that fusion with a cell membrane or liposome integrity is affected by change in temperature and/or pH (see, e.g. U.S. Pat. Nos. 4,789,633; 4,873,089; 6,200,598; and 6,726,925; incorporated herein by reference). Generally, pH sensitive liposomes are acid sensitive. Thus, fusion is enhanced in physiological environments where the pH is mildly acidic, for example the environment of a lysosome, endosome and inflammatory tissues. This property allows direct release of the liposome contents into the intracellular environment following endocytosis of liposomes (Mizoue, T., 2002, Int. J. Pharm. 237:129-137).

Liposomes also include vesicles derivatized with a hydrophilic polymer, as provided in U.S. Pat. Nos. 5,013,556 and 5,395,619, hereby incorporated by reference, (see also, Kono, K. et al., 2000, J. Controlled Release 68:225-35; Zalipsky et al., 1995, Bioconjug. Chem. 6:705-708) to extend the circulation lifetime in vivo. Hydrophilic polymers for coating or derivation of the liposomes include polyethylene glycol, polyvinylpyrrolidone, polyvinylmethyl ether, and polyaspartamide. Other types of suitable coatings will be apparent to the skilled artisan.

Liposomes are prepared by ways well known in the art (see for example, Szoka et al., 1980, Ann. Rev. Biophys. Bioeng. 9:467-508). One typical method is the lipid film hydration technique in which lipid components are mixed in an organic solvent followed by evaporation of the solvent to generate a lipid film. Hydration of the film in aqueous buffer solution results in an emulsion, which may be sonicated or extruded to reduce the size and polydispersity. Other methods for forming liposomes include reverse-phase evaporation (sec, e.g., Pidgeon et al., 1987, Biochemistry 26:17-29; Duzgunes et al., 1983, Biochim. Biophys. Acta. 732:289-99), freezing and thawing of phospholipid mixtures, and ether infusion.

for topical administration, the compositions may be formulated as solutions, gels, ointments, creams, suspensions, etc. as are well-known in the art. In some embodiments, the conditioned media may be applied via transdermal delivery systems, which slowly releases the active compound for percutaneous absorption. Permeation enhancers may be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Moreover, the conditioned media components may be partially entrapped in the particulate polymeric matrix upon formation thereof. Mild particulate formation conditions, such as those employed by Cohen et al., 1991, Pharmaceutical Research 8:713-720 may be used to retain the activity of the factors in the conditioned media. Other polymeric particulate dosage forms (e.g. non-biodegradable dosage forms) will be apparent to the skilled artisan. In embodiments where three dimensional tissues are administered, the three dimensional tissues may be suspended in serum free culture medium, basal culture media, complex culture media, or balanced salt solutions. In other embodiments, the media may contain pharmaceutically acceptable additives, such as vitamins, inorganic salts, amino acids, carbon sources, fatty acids, buffers, and serum. Non limiting examples of media and diluents include phosphate buffered saline, Hank's Balanced Salt Solution, Earle's salts, Modified Eagles Medium, Dulbecco's Modified Eagles Medium, RPMI medium, Iscove's medium, and Leibovitz L-15. Resuspension or replacement with fresh cell medium may be done shortly before administration of the three dimensional tissues.

In other embodiments, the three dimensional tissues are cryopreserved preparations, which are thawed prior to use. Pharmaceutically acceptable cryopreservatives include, among others, glycerol, saccharides, polyols, methylcellulose, and dimethyl sulfoxide. Saccharide agents include monosaccharides, disaccharides, and other oligosaccharides with glass transition temperature of the maximally freeze-concentrated solution (Tg) that is at least −60, −50, −40, −30, −20, −10, or 0° C. An exemplary saccharide for use in cryopreservation is trehalose. Cryopreservation is used not only for storage purposes but may also be carried out to increase the production of growth factors (U.S. Pat. No. 6,291,240).

In some embodiments, the three dimensional tissues are treated to kill the cells prior to use. In some embodiments, the extracellular matrix deposited on the scaffolds may be collected and processed for administration (see U.S. Pat. Nos. 5,830,708 and 6,280.284, incorporated herein by reference). In other embodiments, the three dimensional tissue in which the cells have been killed, and thus lack viable cells, may be administered to promote growth of hair.

In other embodiments, the three dimensional tissue may be concentrated and washed with a pharmaceutically acceptable medium for administration. Various techniques for concentrating the compositions are available in the art, such as centrifugation or filtering. Exemplary techniques include as non-limiting examples, dextran sedimentation and differential centrifugation. Formulation of the three dimensional tissues may also involve adjusting the ionic strength of the suspension to isotonicity (i.e., about 0.1 to 0.2) and to physiological pH (i.e., pH 6.8 to 7.5). The formulation may also contain lubricants or other excipients to aid in administration or stability of the cell suspension. These include, among others, saccharides (e.g., maltose) and organic polymers, such as polyethylene glycol and hyaluronic acid. Additional details for preparation of various formulations are described in U.S. Patent Publication No. 2002/0038152, incorporated herein by reference.

The compositions above may be used alone, or in combinations with other compatible hair promoting compounds. In some embodiments, as further described above, the compositions may be used adjunctively with other compounds or compositions that promote grow of hair. In some embodiments, the adjunctive agent comprises an agent that induces skin vascularization. In some embodiments, the inducer of skin vascularization is VEGF. Effect of VEGF on hair growth appears to derive from increased blood flow to the hair follicle. VEGF may be injected or administered topically with skin penetration enhancers. In other embodiments, the inducer of skin vascularization is a modulator of VEGF activity, such as 6-(1-piperidinyl)pyrimidine-2,4-diamine 3-oxide (i.e., minoxidil), available under the tradename Rogaine®. Minoxidil is a vasodilator and was originally developed as an oral drug to treat high blood pressure. Minoxidil, however, appears to induce VEGF activity when applied to the skin, thereby increasing vascularization in the treated area.

In some embodiments, the compositions are used adjunctively with an agent that decreases level of dihydrotestosterone in the skin. This steroid hormone is elevated in subjects with androgenetic alopecia and is known to decrease the anagen stage of hair follicle growth. In some embodiments, the agent used for decreasing dihydrotestosterone levels in the skin is an inhibitor of 5 α-reductase Type II, which is the intracellular enzyme responsible for conversion of testosterone to dihydrotestosterone. Inhibiting this enzyme leads to a decrease in hormone levels and subsequent increase in the time period of anagen. Exemplary 5 α-reductase Type II inhibitor include, among others, [5-,17-N-(1,1-dimethylethyl)-3-oxo-4-azaandrost-1-ene-17-carboxamide (i.e., finasteride), available under the tradename Propecia® and dutasteride, available under the tradename Avodart®. Other 5 α-reductase inhibitors will be apparent to the skilled artisan (see, e.g., U.S. Pat. Nos. 6,696,484; 6,380,179; and 6,015,806; publications incorporated herein by reference).

### 5.7 Treatment of Hair Loss

The conditioned media, or components thereof, such as isolated Wnt proteins, or three dimensional tissues find uses in enhancing hair growth in subjects where additional hair growth is desirable. In these embodiments, the compositions are useful for cosmetic applications and for treating conditions of hair loss.

In some embodiments, the compositions have cosmetic applications for enhancing growth of hair in areas where a higher density of hair follicles is desirable. As disclosed herein, the hair promoting effect of the conditioned media is generally localized to the site of injection. This allows sculpturing of the areas for enhancement by restricted application to those areas where additional hair growth is desired. Exemplary localized cosmetic enhancements include the eyebrow, hairline, or scalp. The compositions may be used to generate fuller and thicker eyebrows, induce hair growth to alter the hairline, or generate higher density of hair in the scalp. Other cosmetic applications will be apparent to the skilled artisan.

In other embodiments, the compositions are used to treat various forms of hair loss, a common problem having many different causes, including age-related, genetic, autoimmune, and environmental factors. In some embodiments, the hair loss is a form of alopecia, various forms of which are classified into scarring and non-scarring alopecia.

In some embodiments, the compositions are used to treat subjects affected with androgenetic alopecia, a type of nonscarring alopecia, which in men is referred to as male pattern baldness and/or age related alopecia. The disorder, however, affects both men and women. The condition is thought to arise from the action of the steroid hormone dihydrotestosterone on genetically susceptible follicles resulting in gradual reduction in the size of the follicles and shortening of the anagen phase. The telogen phase remains constant, with the end result being an area denuded of hair. Androgenetic alopecia is an inherited condition, affecting about 25% of men before the age of 30 and two-thirds of all men before the age of 60. Female androgenetic alopecia is more diffuse and less patterned than the forms seen in men. In females, estrogens may protect the follicles from androgen effects to some extent such that an acceleration of hair loss is often noted after menopause.

In other embodiments, the compositions are used to treat alopecia areata, a disorder that causes sudden hair loss on the scalp and other regions of the body. In alopecia areata, an autoimmune reaction attacks the hair follicles, resulting in the arrest of the hair growth stage. Although the hair is most subjects grow back without treatment, up to 10% of cases result in chronic or recurrent baldness. Conventional treatment for alopecia areata is directed to limiting the autoimmune reaction by administering an immunosuppressive steroid (e.g., Cortisol) into the affected area. In addition, minoxidil may be used to promote hair regrowth. In the embodiments herein, the conditioned media may be administered to subjects affected by alopecia areata to hasten the regrowth of hair for acutely affected patients or to induce hair growth in chronic sufferers of the malady.

In further embodiments, the compositions used to treat chemically induced alopecia. Most hair loss associated with chemical exposure is alopecia induced by treatment with chemotherapeutic agents, such as cytotoxic agents used for treatment of cell proliferative disorders, various neoplasms, and bone marrow transplantation. Severity typically depends on the type of drug, the dose, and its mode of administration. Although not all chemotherapeutic agents cause hair loss and not all hair loss is permanent, some systemically administered cyotoxic agents, such as busulfan, can lead to permanent hair loss or sparse regrowth following termination of the therapy. This loss may result from hair follicle stem cell destruction or from acute damage to the keratinocytes of the lower portion of some follicles. Treatments using the compositions may result in recruitment of epidermal stem cells and enhancement of the differentiation and growth of new hair follicles.

In still other embodiments, the compositions are used to treat radiation induced alopecia. Only hair that is in a radiation treatment field will be affected with hair loss. Generally, the hair loss will begin approximately 2-3 weeks after the start of treatments. This hair will grow back after the treatments are completed. However, when a higher dose of radiation is delivered, there is a chance that the hair loss will be permanent. The compositions disclosed herein may have applications for enhancing hair growth in the radiation damaged area.

In some embodiments, the compositions are used to treat subjects who are either undergoing or have undergone hair transplantation. The use of hair transplantation is based on the observation that hair retains the characteristics from where it is taken and does not take on new characteristics from where it is placed. Generally, a thin strip of hair and scalp obtained from the back of the head is cut into smaller clumps of five or six hairs. Tiny cuts are made in the balding area and a clump is implanted into each slit. Minigrafts, micrografts, or implants of single hair follicles are used to fill in between larger implant sites and can provide a more natural-looking hairline. Larger grafts provide increased hair density where needed. In the embodiments herein, the compositions may be used adjunctively with the hair transplantation to promote hair growth following transplant, and/or preoperatively to prime the area for receiving the hair follicle transplant.

In still other embodiments, the compositions are used in vitro to promote differentiation of epidermal stem cells that give rise to hair follicle cells and/or promote development of hair follicles in culture. Generally, hair follicles or epidermal stems in culture are contacted with the conditioned media made from the three dimensional tissues. Such culture systems may be used to growth hair follicle cells for purposes of screening agents that modulate hair follicle growth or identify factors involved in hair follicle development. Various cultures systems use in art are described above.

### 5.8 Administration and Dosages

As discussed above, the conditioned medium may be used directly or processed in combination with a pharmaceutically acceptable excipients, vehicles, and carriers. For promoting growth of hair, the compositions or various pharmaceutical compositions thereof are administered in a manner such that the cells that form or produce the hair follicle (dermal papilla or epidermal stems cells) are contacted with the conditioned medium or factors produced by the three dimensional tissues. Generally, the compositions are administered intradermally and/or subcutaneously. As used in the art, “intradermal” administration refers to administration in or into the skin. In some embodiments, the intradermal administration is injection of a small volume into the upper layer of skin (i.e., the dermis), just beneath the epidermis. The dermis is composed of three types of tissue that are present throughout. These tissues include collagen, elastic tissue, and reticular fibers. Administration may be to any layer within the dermis.

“Subcutaneous” administration refers to administration just beneath the skin (i.e., beneath the dermis). Generally, the subcutaneous tissue is a layer of fat and connective tissue that houses larger blood vessels and nerves. The size of this layer varies throughout the body and from person to person. As used herein, the interface between the subcutaneous and muscle layers is to be encompassed by subcutaneous administration.

In other embodiments, the compositions may be injected intramuscularly, just beneath the subcutaneous layer. This mode of administration may be feasible where the subcutaneous layer is sufficiently thin so that the factors present in the compositions can migrate or diffuse from the locus of administration and contact the epidermal stem cells and hair follicle cells responsible for hair formation. Thus, where intradermal administration is contemplated, the bolus of composition administered is localized proximate to the subcutaneous layer.

It is to be understood that administration of the compositions is not restricted to a single route, but may encompass administration by multiple routes. For instance, exemplary administrations by multiple routes include, among others, a combination of intradermal and intramuscular administration, or intradermal and subcutaneous administration. Multiple administrations may be sequential or concurrent. Other modes of application by multiple routes will be apparent to the skilled artisan.

In some embodiments, the compositions may be administered topically as an adjunct to the intradermal, subcutaneous, or intramuscular administrations. Topical applications may have the effect of increasing vascularization in the applied region, as well as providing some source of growth factors that are involved in promoting hair growth. The effect of VEGF present in the topically applied conditioned media combined with the direct administration of the conditioned media via intradermal, subcutaneous, or intramuscular routes may provide a better effect in enhancing the growth of hair than injection of the conditioned media in the absence of topical administration.

### 5.9 Dosage

The compositions or active components thereof, will generally be used in an amount effective to treat or prevent the particular disease being treated. The compositions may be administered therapeutically to achieve therapeutic benefit or prophylactically to achieve prophylactic benefit. By therapeutic benefit is meant eradication or amelioration of the underlying condition or disorder being treated, e.g., amelioration of the underlying hair loss. Therapeutic benefit also includes halting or slowing the progression of the disease, regardless of whether improvement is realized.

For prophylactic administration, the active compound may be administered to a patient at risk of developing a disorder characterized by, caused by or associated with hair loss, such as the various disorders described herein. For example, the compositions may be administered prior to appearance of symptoms of hair loss or after the first signs of hair loss to limit worsening of the condition. Prophylactic administration may be applied to avoid the onset of symptoms in a patient diagnosed with the underlying disorder. Active compounds may also be administered to healthy individuals where the administration is for cosmetic purposes.

The amount of the composition administered will depend upon a variety of factors, including, for example, the type of composition, the particular indication being treated, the mode of administration, whether the desired benefit is prophylactic or therapeutic, the severity of the indication being treated and the age and weight of the patient, and effectiveness of the dosage form. Determination of an effective dosage is well within the capabilities of those skilled in the art.

Initial dosages may be estimated initially from in vitro assays. For example, initial dosages may be formulated using organ culture of dermal papilla or whole hair follicles (Philpott et al., 1996, Dermatol Clin. 14(4):595-607; Jahoda et al., 1993, J Invest Dermatol. 101(1 Suppl):33S-38S. Initial dosages can also be estimated from in vivo data, such as animal models. Animals models useful for testing the efficacy of compositions for enhancing hair growth include, among others, rodents, primates, and other mammals. The skilled artisans can determine dosages suitable for human administration by extrapolation from the in vitro and animal data.

Dosage amounts will depend upon, among other factors, the activity of the conditioned media, the mode of administration, the condition being treated, and various factors discussed above. Dosage amount and interval may be adjusted individually to provide levels sufficient to maintain therapeutic or prophylactic effect of enhancing growth of hair. The compositions may be administered one time daily, two times daily, or once per week depending upon, among other things, the indication being treated, the level of hair growth desired, and the judgment of the prescribing physician. The compositions will provide therapeutic or prophylactic benefit without causing substantial toxicity or adverse immunological reaction. Skilled artisans will be able to optimize effective local dosages without undue experimentation.

The density of administration (e.g., local administration for a defined surface area) may depend on the degree of hair loss, the level of hair growth desired, the volume of conditioned medium administered, the efficacy of the composition, and the density fair follicles typically present in the area. In some embodiment, the number of administrations per cm² is about 1-2 localized applications, up to about 5 localized applications, and up to about 10 localized applications within the defined surface area. The surface pattern of administrations is done to produce the desired pattern of hair growth. Patterns may be random or ordered, such as evenly spaced columns and/or rows. Applications of the conditioned medium may also follow the contours of the desired hairline, such as the presence of hair on the eyebrow or scalp.

### 5.10 Kits

Further provided herein are kits comprising the compositions in various forms, as described herein. The kit may contain liquid, solid, or powder formulations of the conditioned media, in format suitable for administration. Suitable diluents for reconstituting the composition may be included in the kits for preparing the composition for administration. In other embodiments, the kits comprise three dimensional tissues suitable for administration into the skin. The kits may further comprise devices for administration, such as injection devices (e.g., manual or electronic driven), for use by the person skilled in the art. Where other compounds are administered adjunctively with the conditioned media, they may also be included in the kits. Accompanying the compositions are instructions for preparing and administering of the compositions. These may be in various formats, such a printed mailer, magnetic disk, magnetic tape, compact disc, flash memory, or other mediums suitable for conveying the appropriate information.

### 6. EXAMPLES

### 6.1 Example 1

### Three Dimensional Tissue Conditioned Medium Promoted Differentiation and Repair of Epithelial Cells

Materials and Methods

Epithelial Cell Growth and Differentiation Assay. This study evaluated the effects of three-dimensional tissue conditioned medium on four different human epithelial cell lines in vitro. The cell lines utilized were as follows: (1) primary human epidermal keratinocytes at passage 2-3 (P2-3), (2) an immortalized intestinal epithelial cell line (Caco-2), (3) an immortalized respiratory epithelial cell line (NCI-H292), and (4) an immortalized colonic epithelial cell line (HT-29). These immortalized cell lines are accepted models of the tissue from which they were derived, and are well characterized in vitro. Epidermal keratinocytes represent a primary cell type.

Cell proliferation. Cell lines were obtained from ATCC and plated at approximately 5000 cells/cm² in 24 well plates and cultured for 2-3 days in the presence of 10% (v/v) test materials or control non-conditioned medium. Primary human epidermal keratinocytes at P2 in KGM-2 medium (Clonetics, Inc.) were also evaluated in some experiments. Controls consisted of untreated cells as well as non-conditioned medium as a negative control. After rinsing briefly in PBS, relative cell number was estimated by DNA fluorescence using Molecular Probes, Inc. CyQuant Cell Proliferation kit, and data is expressed as relative DNA fluorescence per well, with 6 wells per condition. Experiments were repeated twice for each cell line. In the studies herein, conditioned medium is referred to as Nouricel.

Immunofluorescence and Phase-contrast Microscopy. Primary human epidermal keratinocytes at passage 2 were cultured in serum free medium supplemented with 10% (v/v) control non-conditioned medium or test substances for 2 days on collagen coated glass chamber slides. Cultures were rinsed, fixed in paraformaldehyde, permeabilized in 0.1% Tween-20 in PBS. Primary antibodies were as follows: Mab×ZO-1 and Rb×Claudin-1 (Zymed, Inc.) at 2 μg/ml each for 30 minutes followed by detection with fluorochrome conjugated secondary antibodies and mounting in Vectashield (Vector Labs, Inc.) with DAPI added as a nuclear counterstain. Live cultures were photographed prior to fixation under phase contrast illumination at approximately 100× magnification.

Transmission Electron Microscopy on Organotypic Cell Cultures. Cells were cultured on collagen coated microporous membranes for 10 days after seeding at high cell density (100,000/cm²), conditions known to support differentiation of the Caco-2 cell line, in the presence of 10% (v/v) of the <10 kD three-dimensional stromal tissue conditioned medium Permeate or an untreated control. Both the Caco-2 and NCI-H292 cell lines were evaluated. Cultures were rinsed and then fixed in modified Karnovsky's fixative, and processed for electron microscopy using standard techniques.

Expression Analysis by Fluorescent Gene Chip Arrays. Single low-density cultures of epidermal keratinocytes, Caco-2 cells, and NCI-H292 cells in 10 cm dishes were treated with concentrated three-dimensional stromal tissue conditioned medium or a non-conditioned medium control for 3-5 days, and mRNA was isolated. After conversion to cDNA and fluorescent labeling, pooled cDNA's were hybridized to gene-chip arrays. Fluorescence detection of hybridized probes to approximately 10,000 known gene transcripts was conducted at UC Irvine and the data was analyzed using Genespring software. Normalized fluorescence was graphed comparing the relative change in expression profiles.

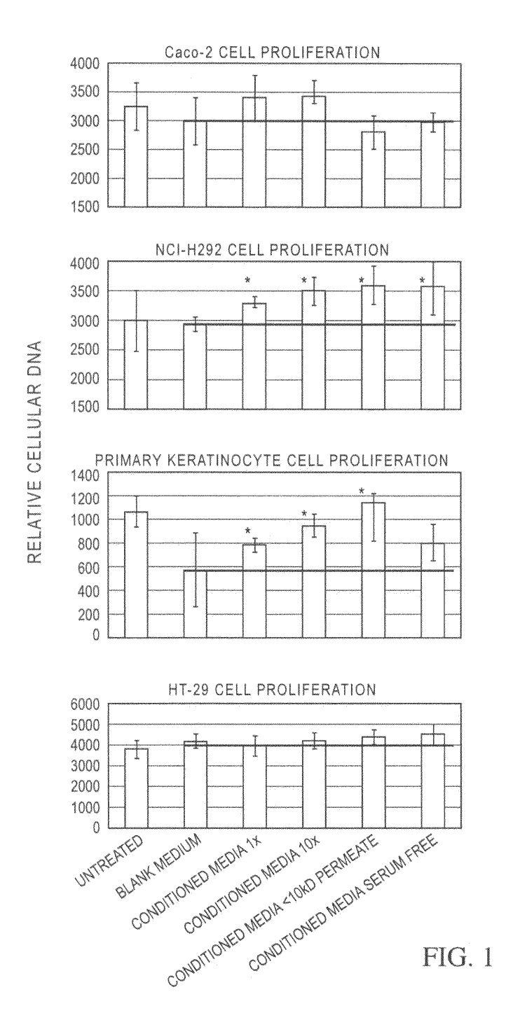

Results. A comparison of the effects of three-dimensional stromal tissue conditioned medium on cell proliferation in four different epithelial cell types is shown in FIG. 1. Not all cell lines responded and not all conditioned medium were effective. The effects of three-dimensional stromal tissue conditioned medium on cell proliferation were cell type-specific in that three-dimensional stromal tissue conditioned medium enhanced cell growth in both primary epidermal keratinocytes and NCI-H292 cells, but not for the most part in Caco-2 or HT-29 cells. When these three-dimensional stromal tissue conditioned medium treated cultures were examined by phase contrast microscopy, again the effects were cell type-specific in that HT-29 cells were not altered morphologically, but keratinocytes (data not shown), Caco-2, and NCI-H292 cells were altered. Both the Caco-2 and NCI-H292 cell lines displayed the formation of dome-like structures when examined by phase contrast microscopy. The Caco-2 cell line also displayed altered localization of both adherens junction (ZO-1) and tight junction markers by immunofluorescence after three-dimensional stromal tissue conditioned medium treatment.

Since the formation of intercellular adherens and tight junctions is a key component of epithelial cell differentiation, cells were evaluated three-dimensional stromal tissue conditioned medium's effects on Caco-2 and NCI-H292 cell lines in high-density organotypic cultures using microporous inserts to promote apical/basolateral differentiation and junction formation. Analysis of these cultures by transmission electron microscopy (TEM) revealed a number of apparent effects of the >10 kD permeate from the three-dimensional stromal tissue conditioned medium concentration process on cellular differentiation.

The apparent formation of dome-like structures in the Caco-2 and NCI-H292 cell lines may indicate an enhancement or induction of differentiation, as mucin-production (a marker of both intestinal and respiratory epithelium) has been reported to lead to similar morphological changes in these cell lines as well as primary human intestinal and respiratory cells in culture.

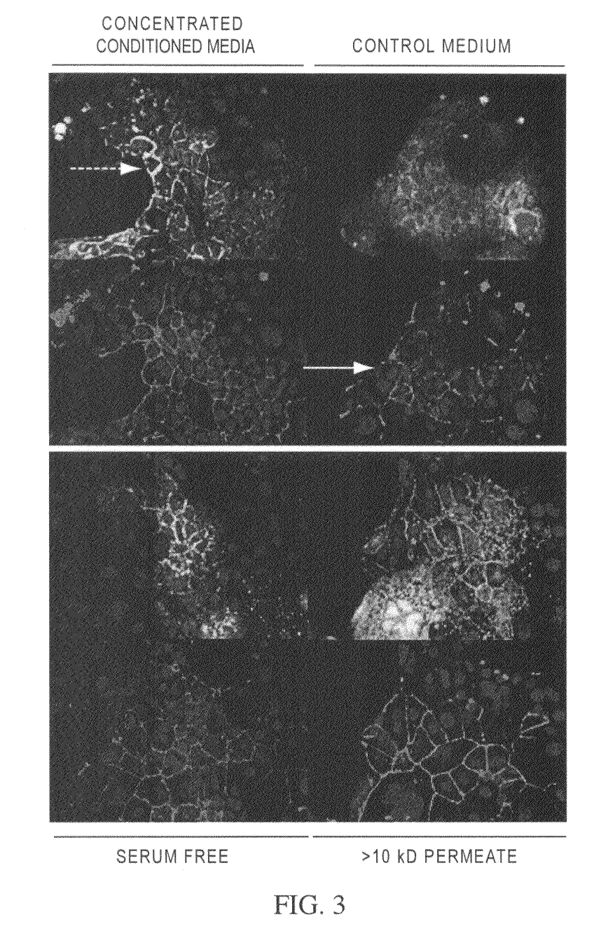

Since the formation of these putative differentiated structures is accompanied by alterations in intercellular junctions, we evaluated the effects of conditioned medium on adherens and tight junction markers in Caco-2 cells by immunofluorescence. The results of this experiment are shown in FIG. 3, which depicts immunofluorescence analysis for adherens (ZO-1) and tight (claudin-1) junction markers in Caco-2 cells treated with three-dimensional stromal tissue conditioned medium on collagen coated glass slides. Note the discontinuous staining for ZO-1 in the control medium panel (white arrow), and the junctional localization of claudin-1 in all the three-dimensional stromal tissue conditioned medium treated panels (dashed white arrow).

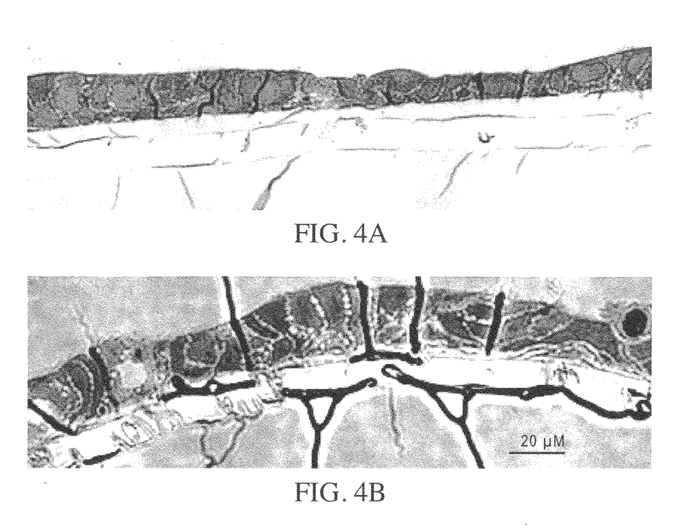

Since simple epithelial differentiation displays number of well-characterized morphological changes that can best be identified by transmission electron microscopy (TEM), and since standard monolayer cultures are not favorable for this differentiation pathway, TEM analysis was performed on three-dimensional stromal tissue conditioned medium treated organotypic, high-density microporous membrane cultures. A thick-section at is shown for control and three-dimensional stromal tissue conditioned medium treated Caco-2 cells (FIG. 4). There is a difference in overall thickness, increased columnar shape, and increased intercellular spaces in cells treated with three-dimensional stromal tissue conditioned medium. These are all characteristics of normal differentiation of these cell types.

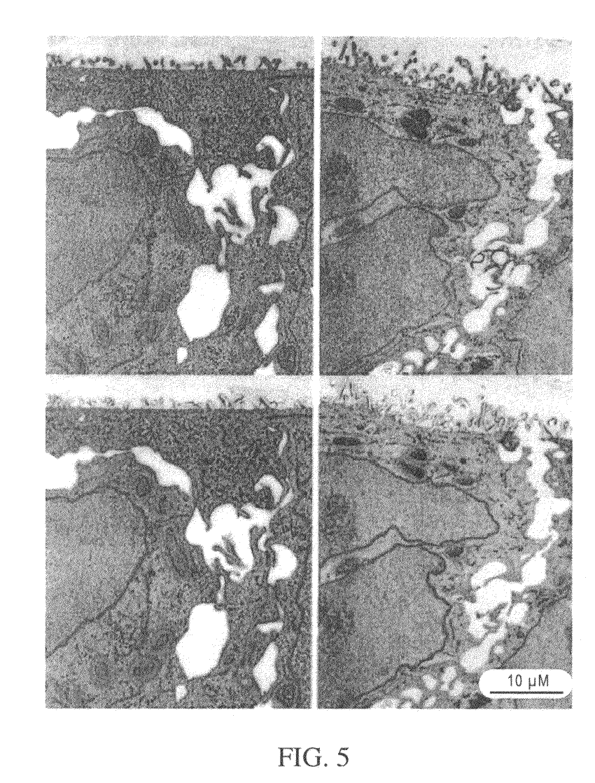

Under much higher magnification, a number of features altered after three-dimensional stromal tissue conditioned medium treatment are observed. FIG. 5, which shows TEM analysis of the effects of conditioned medium <10 kD permeate on Caco-2 cells in high density organotypic cultures. The lower duplicate panels have highlighted the following features: nuclear membranes, brushborder microvilli, mitochondria, and cellular processes. Note the increase in cellular processes, microvilli, and mitochondrial location (apical in the three-dimensional stromal tissue conditioned medium sample). Although not highlighted, tight junctions were less frequent in the conditioned medium sample than the control.

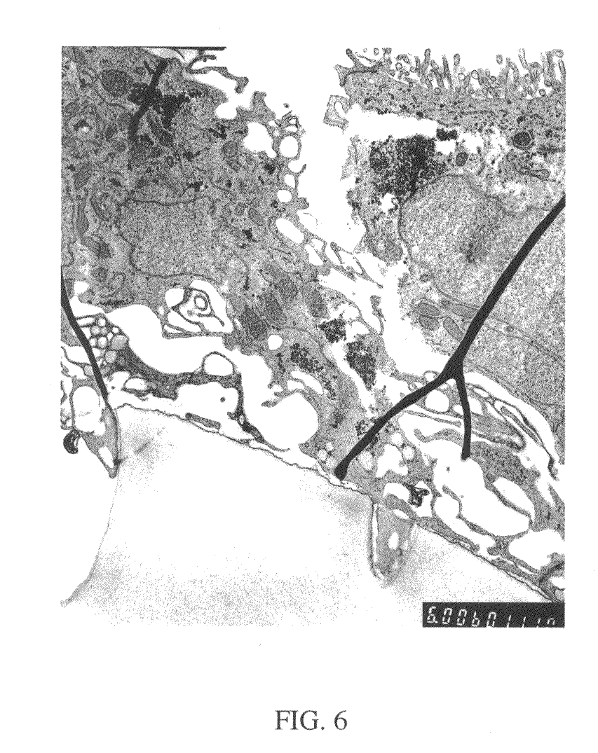

FIG. 6 is a TEM analysis of effect of conditioned medium <10 kD permeate on Caco-2 cells in high density organotypic cultures showing effects on cellular processes, apical microvilli, and dense glycogen deposits. Micrographs show the high degree of cellular processes apparently induced by three-dimensional stromal tissue conditioned medium in these cultures of Caco-2 cells. Glycogen deposits and dense microvilli can also be seen.

In a preliminary screen, the effects of concentrated three-dimensional stromal tissue conditioned medium on global gene expression were examined for cultures of epidermal keratinocytes, Caco-2 cells, and NCI-H292 cells (as a supplement). When compared to a non-conditioned medium control, three-dimensional stromal tissue conditioned medium altered the expression level of genes in three cell types (epidermal keratinocytes, Caco-2 cells, and NCI-H292 cells), although in the relative rank order of keratinocytes>NCI-H292>>Caco-2 cells.

The results above suggest that the conditioned medium contains activity (or activities) that can affect some epithelial cells in vitro. Three-dimensional stromal tissue conditioned medium can enhance cellular proliferation of epithelial cells in a cell line-specific manner, although not all versions of conditioned medium are effective. Three-dimensional stromal tissue conditioned medium can also alter epithelial cell morphology in both low-density monolayer and organotypic cell cultures at both the light and electron microscopic levels. Some of these changes are consistent with enhanced differentiation.

### 6.2 Example 2

### Injection of Stromal Tissue Conditioned Medium in a Mouse Hair Model

This study evaluated the effects of conditioned medium from three dimensional tissue comprised of fibroblasts on hair follicle development in C57B1/6 mice. The experiments sought to examine whether the medium contained factors mimicking inductive signals from dermal papilla cells to induce hair growth. Candidates for hair growth activity induced by dermal fibroblast conditioned medium include Wnt gene products because (1) Wnt-signaling occurs through nuclear β-catenin during fetal development; (2) stabilized β-catenin mutant transgenes form pilosebaceous tumors in mice and humans, and (3) transplanted dermal papilla can induce follicles in rodents and this requires Wnt-signals. Also, anagen induction is thought to involve KGF/FGF-7, hormones (T3, PTH, androgens), sonic hedgehog, and Wnt gene products.

C57B1/6 mice are suitable for hair studies as they exhibit synchronized hair follicle cycling, an extended telogen phase from about days P45 to P65, and exhibit follicular melanocytes in bulb only during anagen such that the start of anagen is visually evidenced by a dark skin color. The study examined the effects of conditioned medium on hair growth by single injection subcutaneously (SQ) in the dorsal skin of approximately 7 week female C57B1/6 mice (telogen at dorsal injection site). Histology and photography were performed at days 14 (all groups) and 30 (control, neat) N=3. Test groups included Blank Medium Control, 10× neat, 10× diluted 1/10, 1/100 permeate form 10 kD concentration, topically applied, and experimental serum-free conditioned medium. The tissues were examined for formation of hair follicles.

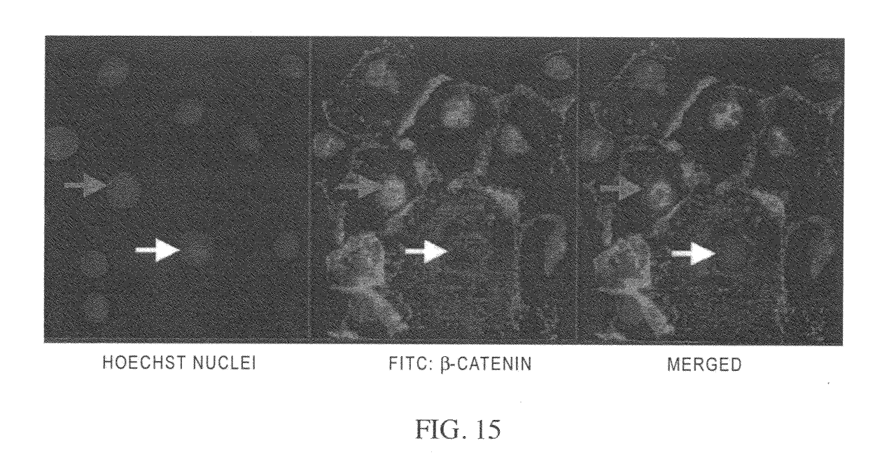

In vitro analyses of Wnt signaling in epidermal keratinocytes in vitro were also performed. FIG. 15 shows Wnt signaling in epidermal keratinocytes in vitro. Nuclear translocation of β-catenin is induced by conditioned medium, providing strong evidence that the conditioned medium contains Wnt proteins.

The results in mice show that conditioned medium made from three dimensional tissues is active inducing hair growth when injected into the skin. Hair follicle development is localized to the injection site. Wnt and Wnt-mediated signaling activity is a possible mechanism behind the hair growth in mice treated with the conditioned media because (a) Wnt genes are expressed by fibroblasts in gene chips, and (b) conditioned medium induces Wnt signaling in epidermal keratinocytes in vitro.

The foregoing descriptions of specific embodiments of the present disclosure have been presented for purposes of illustration and description. They are not intended to be exhaustive or to limit the scope of the disclosure to the precise forms disclosed, and obviously many modifications and variations are possible in light of the above teaching.

All patents, patent applications, publications, and references cited herein are expressly incorporated by reference to the same extent as if each individual publication or patent application was specifically and individually indicated to be incorporated by reference.

1. A method of promoting growth of hair, comprising:
administering intradermally or subcutaneously to a subject an effective amount of a composition comprising conditioned medium made from a three dimensional tissue.

2. The method of claim 1 in which the living cells comprise fibroblasts.

3. The method of claim 2 in which the fibroblasts are dermal fibroblasts.

4. The method of claim 1 in which the three dimensional tissue comprises a biocompatible, non-living material.

5. The method of claim 4 in which the biocompatible, non-living material comprises a biodegradable material.

6. The method of claim 5 in which the biodegradable material is polyglycolic acid, polylactide, polylactide-co-glycolic acid, catgut sutures, cellulose, gelatin, collagen, or dextran.

7. The method of claim 1 in which the biocompatible, non-living material comprises a non-biodegradable material.

8. The method of claim 7 in which the non-biodegradable material is polyamide, polyester, polystyrene, polypropylene, polyacrylate, polyvinyl, polycarbonate, polytetrafluoroethylene, nitrocellulose, or cotton.

9. The method of claim 1 in which the three dimensional tissue comprises a mesh.

10. The method of claim 1 in which the subject is afflicted with alopecia.

11. The method of claim 10 in which the alopecia is androgenetic alopecia.

12. The method of claim 10 in which the alopecia is alopecia areata.

13. The method of claim 10 in which the alopecia is chemotherapy induced alopecia.

14. The method of claim 10 in which the alopecia is radiation induced alopecia.

15. The method of claim 10 in which the alopecia is age-related alopecia.

16. The method of claim 1 in which the hair growth is that of a transplanted hair follicle.

17. The method of claim 1 in which the composition is administered adjunctively with at least one agent that induces skin vascularization.

18. The method of claim 17 in which the agent is minoxidil.

19. The method of claim 17 in which the agent is vascular endothelial growth factor (VEGF).

20. The method of claim 1 in which the composition is administered adjunctively with at least one agent that decreases levels of dihydrotestosterone in the skin.
